(19) United States
(12) Patent Application Publication
(10) Pub. No.: US 2023/0255961 A1
(43) Pub. Date: Aug. 17, 2023

(54) Rotomeric Isomers of 4-Alkyl-5-Heteroaryl-3H-1,2-Dithiole-3-Thiones

(71) Applicant: ST IP Holding AG, Zug (CH)

(72) Inventor: Bomi FRAMROZE, Menlo Park, CA (US)

(21) Appl. No.: 17/946,789

(22) Filed: Sep. 16, 2022

Related U.S. Application Data
(63) Continuation of application No. PCT/IB2021/000153, filed on Mar. 16, 2021.
(60) Provisional application No. 62/990,352, filed on Mar. 16, 2020.

(51) Int. Cl. A61K 31/497

(52) U.S. Cl. CPC A61K 31/497

(57) ABSTRACT

This disclosure provides, among other things, rotomeric isomers of 4-alkyl-5-heteroaryl-3H-1,2-dithiole-3-thiones, complexes of such isomers in which an individual rotomeric form is obtained by stabilization, and compositions comprising an excess of an individual rotomer (a rotomeric excess) as a stabilized complex. The disclosure also relates to methods of making and using: such rotomers and complexes, including methods of treating a human or animal patient with such rotomers or complexes thereof, e.g., to prevent, treat or reduce the symptoms of various disorders such as mucositis, viral infection, stroke injury, ischemia/reperfusion injury, cardiac ischemia and/or provide protection against oxidative damage in various organs and tissues for a medical purpose.

This application claims the benefit of priority to U.S. Provisional Pat. Appln. No. 62/990,352, filed on Mar. 16, 2020.

### FIELD

This disclosure relates, among other things, to rotomeric isomers of 4-alkyl-5-heteroalyl-3H-1,2-dithiole-3-thiones, complexes of such isomers in which an individual rotomeric form is obtained by stabilization, and compositions comprising an excess of an individual rotomer (a rotomeric excess) as a stabilized complex. The disclosure also relates to methods of making and using: such rotomers; complexes in which an individual rotomer is obtained by stabilization and present in a rotomeric excess; compositions (including rotomeric pharmaceutical compositions) comprising the rotomers or complexes thereof; methods of regulating the function of human oxidation protective genes by using such rotomers or complexes thereof; and methods of treating a human or animal patient with such rotomers or complexes thereof (e.g., to prevent, treat or reduce the symptoms of various disorders such as mucositis, and/or provide protection against oxidative damage in various organs and tissues for a medical purpose).

The disclosure herein relates to new pharmaceutical formulations, compositions and therapies comprising a population of 4-methyl-5-(pyrazin-2-yl)-3H-1,2-dithiole-3-thione (also known as oltipraz) rotomers enriched in an individual rotomer that may be stabilized in a complex.

### BRIEF DESCRIPTION OF THE DRAWINGS

FIG. 1 depicts oltipraz (4-alkyl-5-(pyrazin-2-yl)-3H-1,2-dithiole-3-thione),

FIG. 2 depicts oltipraz shown in a configuration where its two rings are almost orthogonal to each other.

FIG. 3 depicts an oltipraz analog wherein the alpha dithiol sulfur atom is substituted with a carbon atom.

FIG. 4A depicts the positive (+) rotomer of oltipraz.

FIG. 4B depicts the negative (−) rotomer of oltipraz.

FIG. 5 depicts the hydroxymaleate anhydride complex of oltipraz.

Atoms in FIGS. 2-5 are shown as circles (spheres) marked with “H” for hydrogen, “S” for sulfur. “N” for nitrogen, or “O” for oxygen. Carbon atoms are shown as unmarked circles (spheres).

FIG. 6 shows the results of caspase 3/7 activation assay and of positive and negative control in rat cortical neurons.

FIG. 7 shows the prevention of OGD induced caspase 3/7 activation in rat cortical neurons by four rotomer complexes (100 μg/ml) along with positive and negative controls.

FIG. 8 shows the results of caspase 3/7 activation assay and of positive and negative control in mouse neonatal primary cardiomyocyte.

FIG. 9 shows the prevention of OGD induced caspase 3/7 activation in mouse neonatal primary cardiomyocyte by four rotomer complexes (100 μg/ml) along with positive and negative controls.

### DISCLOSURE

4-methyl-5-(pyrazin-2-yl)-3H-1,2-dithiole-3-thione, also known as oltipraz, has been disclosed in both the crystalline and amorphous forms. Oltipraz has been described as having various properties, e.g., as an anti-microbial compound that controls schistosomiasis, as an anti-tumor agent, as a chemo-protectant, and as a compound to help control chemo-radiation induced mucositis.

The mechanism of action of the chemo-protective and mucositis controlling effect has been described to be via the up-regulation of the transcription factor, nuclear factor erythroid 2-related factor 2 (Nrf2) protective genes. Nrf2, which is activated by reactive oxygen species (ROS), appears to be a key regulator in oxidative stress gene regulation. Nrf2 is a member of the Cap'n'Collar family of bZIP proteins. Under normal conditions, Nrf2 is bound to its inhibitor, the cytoskeleton-associated protein Keap1, which represses Nrf2 by facilitating its proteasomal degradation. Upon treatment by antioxidants, Nrf2 is released from Keap1 and it initiates the up-regulation of over 2000 antioxidant genes.

Oltipraz has been shown to activate a series of oxidation protective genes in in vitro gene expression assays using pooled human gingival epithelial cells. Gene expression has also been used to show 200 differentially expressed oxidative genes in subjects with COPD (chronic obstructive pulmonary disorder) when compared with healthy smokers and the significant changes in oxidant response genes observed in vivo were reproduced in vitro using primary bronchial epithelial cells from the same donors. [Pierrou, S., Broberg, P., O'Donnell, R. A., Pawlowski, K. Virtala R., Lindqvist, E. (2007) Expression of Genes Involved in Oxidative Stress Responses in Airway Epithelial Cells of Smokers with Chronic Obstructive Pulmonary Disease. Am. J. of Respiratory and Critical Care Medicine, 175(6), 577-587].

In vitro gene expression has also been studied in human corneal endothelial cells (HCECs) to determine if nuclear oxidative DNA damage increases with age. HCECs respond to this damage by upregulating 4 of 84 of their oxidative stress and DNA damage-signaling genes in an age-dependent manner. [Joyce, N. C., Harris, D. L., Zhu, C. C. Age-Related Gene Response of Human Corneal Endothelium to Oxidative Stress and DNA Damage (2011) Cornea, 52(3), 1641-1649].

Hence, it is widely accepted that gene expression changes observed in in vitro cellular assays have been shown to be representative of physiological oxidation protection conferred to specific organs and tissues.

The chemical structure of oltipraz was optimized for schistosomiasis control by Quantitative Structure Activity Relationship (QSAR) studies. Although oltipraz has recently shown Nrf2 up-regulating activity, whether it is the optimum structure or in an optimal configuration for Nrf2 up regulation, is unknown.

As discussed below, it has been discovered that stable compositions comprising two different rotomers of oltipraz can be prepared . The compositions may comprise up to about an 80%, 85%, 90%, 95%, 97% 98% or 99% rotomeric excess of one of the two rotomers, which may be stabilized in the form of an oltipraz-complex. Compositions comprising higher rotomeric excess percentages, e.g., above 90% (i.e., 95% of one rotomer and 5% of the other) may be less stable at higher temperatures because of the relatively low barrier to rotation. As used herein an “oltipraz-complex” means oltipraz dissociably associated (covalently or non-covalently) with a molecule or atom. Oltipraz-complexes may comprise salts of oltipraz. Oltipraz-complexes may also comprise complexes that are non-salts (not salts) of oltipraz, such as those formed through Vander Waals forces, TE-7t interactions, hydrophobic effects, and the like. Such compositions comprising a rotomeric excess of either of the individual rotomers may specifically increase the up-regulation of the transcriptional factor Nrf2 effect, and hence provides oxidation protection from chemo-radiation and immunotherapy induced mucositis in the gastro-intestinal system, including in the oral cavity.

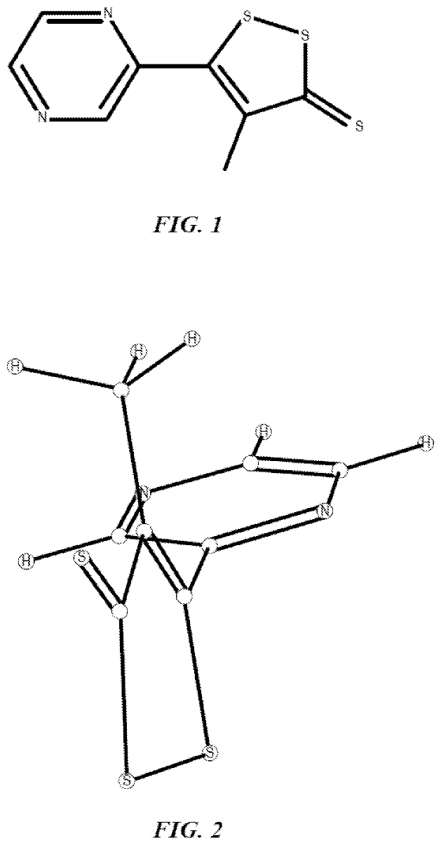

Oltipraz (4-alkyl-5-(pyrazin-2-yl)-3H-1,2-dithiole-3-thione) consists of two rings, the pyrazinyl ring and the dithiolethione ring, connected by a carbon-carbon single bond (FIG. 1).

Normally, free rotation is possible around such a carbon-carbon (C—C) bond such that no specific orientation of the two rings is locked and the two rings have been described in an X-ray structure publication as being in a twisted planar configuration in which conjugation between the unsaturation bonds in each ring allows for the lowest energy stable state, as shown in FIG. 1 above. See, Wei, C. H., Structure of 4-Methyl-5-(2-pyrazinyl)-3H-1,2-dithiole-3-thione (Oltipraz), A New Antischistosomal Drug. Acta Cryst. (1983). C39, 1079-1082)

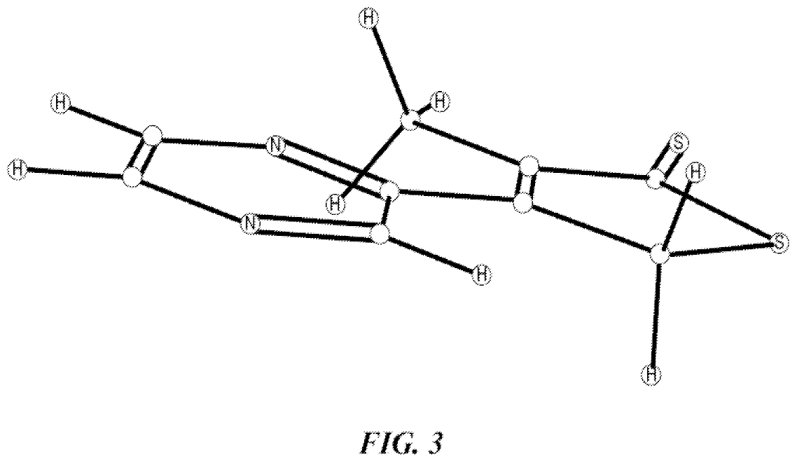

Using energy minimization computer simulations with the Tinker(R) tool and common parameter sets such as Amber and MMFF applied, it was discovered that molecules of oltipraz may also be found in a stable configuration where the two rings are almost orthogonal to each other, e.g., as shown in FIG. 2.

Further computer analysis on analog structures showed that this preferred orthogonal configuration is likely due to sulfur hyperconjugation. Substitution of the alpha dithiol sulfur atom with a carbon atom, which is unable to participate in hyperconjugation, resulted in a planar structure as the minimum energy configuration, with free rotation around the connecting C—C single bond as shown in FIG. 3,

The simulation also showed that an unexpected, unusually high energy barrier exists between one orientation of the two orthogonal rings versus its mirror image, of 14.1 kJ/mol. This may be attributed to the sulfur hyperconjugation effect which needs to be broken to allow rotation along the bond between the two rings. Accordingly, the rotomers are a pair of atropisomers, due to hindered rotation about the bond between the two rings of oltipraz. The Rotomer Compositions

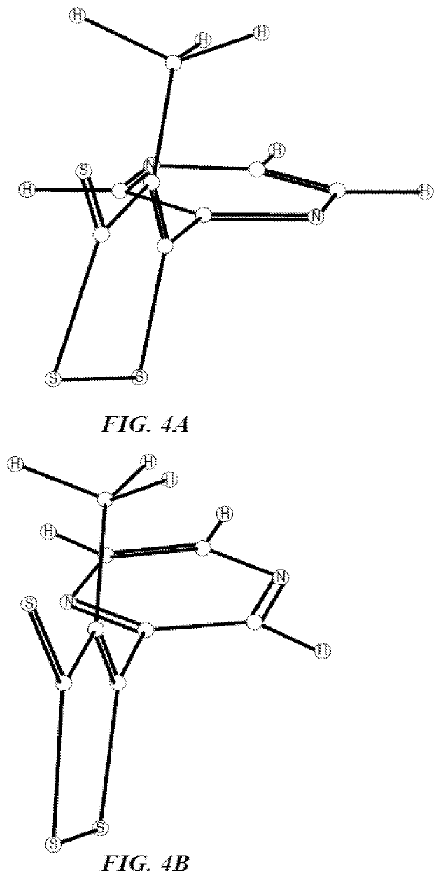

Two distinct rotomers for oltipraz are thus possible. Each of the two rotomers can be defined as two rings that are orthogonal to each other, the (+)-rotomer is defined by orienting the thione and methyl group on top and the alpha nitrogen of the pyrimidine ring on the right, while the (−)-rotomer has the alpha nitrogen of the pyrimidine ring on the left side (see FIGS. 4A and 4B).

It has been found that preparing an oltipraz-complex in the form of a number of salts as described below yields a composition in which the (+)-rotomer (FIG. 4A) can be present in a rotomeric excess (e.g., up to about 80%). As used herein, the term “rotomeric excess” means the excess of one rotomer over the other as is commonly applied to the term “enantiomeric excess”. Thus “rotomeric excess” herein is described as a measurement of the purity of the population of oltipraz rotomers as a whole. It reflects the degree to which a sample contains one rotomer in greater amounts than the other. A meso mixture has a rotomeric excess of 0%, while a single completely pure rotomer has a rotomeric excess of 100%. So, for example, a sample with 70% of one rotomer and 30% of the other has a rotomeric excess of 40% (70%-30%).

The relative percent of one isomer over the other can be assigned using the relative intensities of the thione peaks at ˜1200 and ˜440 cm−1 in an FTIR analysis. The (+) rotomer displays FTIR thione peaks between 420 to 424 and 1210 to 1215 whereas the (−) rotomer shows FTIR thione peaks between 432 to 436 and 1200 to 1205.

Examples of the complexes of oltipraz that may exhibit a rotomeric preference for the (+)-rotomer (see Examples 1 and 3-6 below) include salts resulting from addition or association of a group (e.g., the reversible addition of HCl, alkyl halides or acyl halides) to oltipraz's pyrazinyl ring (e.g., at the alpha nitrogen) yielding a N-substituted pyrazinium ion (with the positive charge most likely centered on the alpha nitrogen of the pyrazinyl group) and an associated anion. Some substituents groups that may be present on a pyrazine ring as the result of salt formation include, but are not limited to hydrogen, alkyl, alkene, alkynyl, acyl, phenacyl or benzoyl, which may be unsubstituted or substituted with other functional groups including, but not limited to halide (e.g., Cl or F), alkyl (e.g., methyl, ethyl, n-propyl, isopropyl), alkoxy (—OCH₃ or —OCH₂CH₃), atyloxy (e.g., -O-phenyl) and the like. For example, such salts may be formed by reacting oltipraz with HCl. In another example, such salts may be formed by reacting oltipraz with an alkyl, alkene, or alkynyl halide such as, methyl chloride, methyl iodide and the like. In another example, such salts may be formed by reaction with an acyl, phenacyl or benzoyl halide (e.g., chloride), such as acetyl chloride, n-butyl chloride, benzoyl chloride and the like.

It also has been found that the (−) rotomer can be prepared (see Examples 2 and 7-11 below) can be present in a substantial rotomeric excess (e.g., up to about 90% or higher, including a 30-40% excess, a 40-50% excess, a 50-60% excess, and a 60-65%, 60-70% and 70-80% excess).

The (−) rotomer oltipraz-complex can be prepared, e.g., as described in Examples 2 and 7-11, by reacting the oltipraz with various carboxy or phosphoric acids and their salts, alkyl and aryl phosphates, alkyl and aryl sulfonic acids and their salts, substituted and unsubstituted thiocarboxy acids and their salts. For example, in the case of the hydroxymaleate oltipraz complex, the oltipraz is reacted with (+)-diacetyl-L-tartaric anhydride in 5 ml of a dry polar solvent such as ultra-dry acetone, followed by the addition of glacial acetic acid. This reaction forms a bond between the alpha nitrogen of the pyrazinyl ring and the oxygen atom of the maleate substantially simultaneously with cyclization rearrangement of the diacetyl tartrate to yield the maleate anhydride ring.

Other complexes of oltipraz that may exhibit a rotomeric preference for the (−) rotomer include those in which a direct bond is formation between the alpha nitrogen of the pyrazinyl ring and an electronegative atom like oxygen, sulfur, or phosphorous that is sufficient to disrupt the hyperconjugation within the molecule. Such oltipraz-complexes may to provide up to 100% rotomeric excess of the (−) rotomer if they can stabilize the (−) rotomer configuration through disruption of the above-described hyperconjugation and/or increased steric strain. For example, such oltipraz-complexes can provide a rotomeric excess of the (−) rotomer in an amount selected from the group consisting of at least 30%, at least 40%, at least 50%, at least 60%, at least 70%, at least 80%, at least 90%, at least 95%, at least 96%, at least 97%, at least 98%, or at least 99%. The rotomeric excess of (−) rotomer thus can be, e.g., an amount selected from the group consisting of a 30-40% excess, a 40-50% excess, a 50-60% excess, a 60-65% excess, a 60-70% excess, a 70-80% excess, an 80-90% excess, and a 90-100% excess. Such oltipraz complexes include those formed by reacting the oltipraz with various carboxy or phosphoric acids and their salts, alkyl and aryl phosphates, alkyl and aryl sulfonic acids and their salts, e.g., substituted and unsubstituted thiocarboxy acids and their salts, e.g., lactic acid. tartronic acid, isothionic acid, isoserine, 2-mercaptoethane sulfonic acid, taurine, propyl phosphonic acid, 2-aminoethylphosphonic acid, diethyl dithiophosphate, diethyl thiophosphate, dimethyl phosphate. It should be noted that it does not appear possible to induce 5-(pyrazin-2-yl)-3H-1,2-dithiole-3-thiones into a configuration like that of oltipraz's (−) rotomer by changing the size of the substituent on the 4-position of the dithiol thione ring (which is a methyl group in oltipraz).

in addition, or in the alternative, to hyperconjugation involving the alpha nitrogen of the pyrazinyl ring, hyperconjugation also can be affected by forming oltipraz-complexes with the meta nitrogen of the pyrazinyl ring. Such (−) rotomeric complexes can be formed via a bond between the meta nitrogen atom and a carbon, oxygen, sulfur or phosphorous atom. This bond can be made to occur singly or together with complexation involving the alpha (ortho) nitrogen atom on the pyrazinyl ring.

As oltipraz conformers are understood to be distributed as a function of their relative energies in a Boltzmann distribution, one means of obtaining a higher rotomeric excess of the individual oltipraz rotomer (or the individual rotomer of an oltipraz-complex) that is most stable under any given set of conditions (e.g., specific solvent), includes cooling a solution of a desired oltipraz composition (e.g., below room temperature such as −78° C. or less) and providing sufficient time for the rotomers to substantially equilibrate. Under such conditions a greater number of molecules will be in more stable (lower energy) conformation(s) than at room temperature. The cooled composition is then dried by evaporation and/or lyophilization, preferably without warming. It has further been found that oltipraz-complexes (e.g., salts) in which either the (−)-rotomer or the (+)-rotomer are in rotomeric excess are able to be substantially purified, e.g., to a purity greater than 90%, greater than 91%, greater than 92%, greater than 93%, greater than 94%, greater than 95%, greater than 96%, greater than 97%, greater than 98%, and greater than 99%. Purification processes and conditions should be chosen so as to minimize the change in the desired rotomeric excess of the composition. For example, the use of temperatures above 40° C. during purification or the use of very polar solvents such as DMSO results in equilibration and substantial loss of the rotomeric excess to close to or equal to zero, whereas the use of chiral chromatography seems to improve rotomeric excess yield. Accordingly, exposing oltipraz-complexes to a chiral media under conditions where one rotomeric form associates with the media more strongly than another rotomeric form, permitting time for the complexes to equilibrate, and eluting the complexes under conditions that do not favor equilibration of the rotomers (e.g., low temperatures), represents a means of enriching for an oltipraz-complex of a desired configuration.

It has further been found that the (+)-rotomer and the (−)-rotomer orientations are not transitional states, but rather are substantially stable configurations when their complexes (e.g., salts) are stored at room temperature such as between 15° C. to 30° C. and 40% to 80% relative humidity, e.g., 20° C. to 25° C. and 50% relative humidity. In this context, “substantially stable” means that the concentrations of the (+)-rotomer and the (−)-rotomer in a composition, as measured by FTIR (as discussed below) within 30 minutes following final purification, will not change by more than an amount selected from the group consisting of 10%, 9%, 8%, 7%, 6%, 5%, 4%, 3%, 2% and 1% when measured using the same FTIR analysis 4 weeks following the final purification.

### Pharmaceutical Compositions Comprising The Rotomer Compositions

The rotomer compositions described above can be prepared as pharmaceutical compositions, either as (i) one or more oltipraz-complexes (e.g.. salts) comprising a rotomeric excess of the (+)-rotomer, (ii) one or more oltipraz-complexes (e.g., the complexes of Examples 2 and 7-11) comprising a rotomeric excess of (−)-rotomer, or (iii) mixtures of (i) and (ii). Pharmaceutical compositions (i)-(iii) above are referred to herein as “rotomer pharmaceutical compositions.” Such rotomer pharmaceutical compositions can be administered in a dry or liquid composition. Formulations of the rotomer pharmaceutical compositions for oral administration also may be presented as a mouthwash, or a carbonated liquid, or an oral spray or aerosol, or an oral ointment, gel, or cream.

In certain aspects, liquids suitable for formulating the rotomer pharmaceutical compositions for oral administration, e.g., buccal administration, may include aqueous and non-aqueous carriers, e.g., water; saline; buffer solutions; organic solvents such as alcohols (e.g., ethanop, glycols, aliphatic alcohols; mixtures of water and organic solvents, and mixtures of organic solvents (optionally also with water), lipid-based materials, polysaccharide-based materials, and other vehicles and vehicle components that are suitable for administration to the oral cavity, as well as mixtures of buccal vehicle components as identified above or otherwise known to the art.

Liquid dosage forms useful for oral administration include pharmaceutically acceptable emulsions, microemulsions, suspensions, syrups and elixirs. In addition to the active ingredients, the liquid dosage forms may contain inert diluents commonly used in the art. Besides inert diluents, the oral compositions can also include adjuvants such as wetting agents, emulsifying and suspending agents (for emulsions and suspensions), sweetening, flavoring, coloring. perfuming and preservative agents.

Alternative aspects of rotomer pharmaceutical compositions suitable for oral administration of the rotomer pharmaceutical compositions include compositions in the form of capsules (including sprinkle capsules and gelatin capsules), sachets, stickpacks, pills. tablets, lozenges, lyophile, powders, granules, implantable compositions, or as a solution or a suspension in an aqueous or non-aqueous liquid, including, e.g., compositions suitable for injection or infusion, or as an oil-in-water or water-in-oil liquid emulsion, or as an elixir or syrup, or as pastilles and/or as mouth washes and the like, each containing a predetermined amount of a composition comprising a quantity of the rotomer pharmaceutical compositions as described herein as the active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), a composition comprising a quantity of the rotomer pharmaceutical compositions can be mixed with one or more pharmaceutically acceptable carriers, and/or any of fillers or extenders, binders, humectants, disintegrating agents solution retarding agents, absorption accelerators, wetting agents, absorbents, lubricants, complexing agents, and coloring agents. The rotomer pharmaceutical compositions may further include components adapted to improve the stability or effectiveness of the formulation. In the case of capsules (including sprinkle capsules and gelatin capsules), tablets and pills, the rotomer pharmaceutical compositions may also comprise buffering agents. Solid compositions of a similar type may also be employed as fillers in soft and hard-filled gelatin capsules using known excipients.

The tablets, and other solid dosage forms of the rotomer pharmaceutical compositions may optionally be scored or prepared with coatings and shells, such as enteric coatings and other coatings well known in the art. They may also be formulated so as to provide slow or controlled release of the active ingredient therein to provide the desired release profile. These compositions may also optionally contain opacifying agents and may be of a composition that they release the active ingredient(s) only, or preferentially, in a certain portion of the gastrointestinal tract, optionally, in a delayed manner. The active ingredient can also be in micro-encapsulated form, if appropriate, with one or more of the above-described excipients.

### Rotomer Pharmaceutical Compositions For Topical Administration

In some aspects, the rotomer pharmaceutical composition may be incorporated into formulations that may be suitable for topical administration, and may include moisturizers, humectants, and other known additives for topical administration. The topical compositions also may be delivered transdermally via a patch that is applied over the skin, and such patches are well known in the art. Persons of skill in the art will recognize other topical delivery compositions and vehicles that may be used.

### Rotomer Pharmaceutical Compositions For Rectal/Colonic Delivery

In certain aspects, the rotomer pharmaceutical compositions described above can be formulated for rectal administration to provide colon-specific delivery using known methods and compositions. Generally speaking, delivery of rotomer pharmaceutical composition via rectal administration route can be achieved by using suppositories, enemas, ointments, creams or foams. Suppositories are among the most common rectal dosage forms, and bases are generally fatty in nature, but water-soluble or water-miscible bases can also be utilized. In order to achieve a desirable bioavailability, the active ingredient should conic in contact with the rectal or colonic mucosa.

Suitable excipients that are known for preparing compositions suitable for rectal administration include, but not limited to, vehicle, preservatives, surfactants, emulsifiers, mineral oils, propellants, thickening agents, lubricants, preservatives, pH adjusting agents, chelating agents, emollients and/or humectants, permeation enhancers, suspension-forming agents or mucoadhesive agents or combinations thereof. The vehicle may include aqueous, non-aqueous or hydroalcoholic components that are known to be compatible with the rectal and colonic mucosa.

Alternatively, colonic absorption can be accomplished through oral administration of the rotomer pharmaceutical compositions, which compositions are designed to release the active oltipraz in the colon. Such compositions can be in an oral dosage form, e.g., a pill, capsule or liquid, that provides delayed release until the dosage form is in the colon

### Rotomer Pharmaceutical Compositions And Devices For Inhalation Administration

In other aspects, rotomer pharmaceutical compositions described above may be delivered via the respiratory tract by providing the composition in inhalable form, e.g., in an inhaler device, either in dry powder form or in a liquid earlier. For example, inhalable compositions can comprise the active rotomer pharmaceutical composition in dry powder compositions provided in dry powder inhalers. See, e.g., WO2014177519 and US20140065219 Alternatively, inhalable compositions can comprise the active ingredient in a liquid carrier such as ethanol. See, e.g., EP2536412 A2.

The disclosure thus also provides a kit comprising (i) a rotomer pharmaceutical composition as described above, and (ii) a device for administering such composition by inhalation. The kit optionally further contains instructions for use.

### Devices For Oral Administration

In certain aspects, liquid formulations of the rotomer pharmaceutical compositions for oral administration may be prepared and administered using a device that facilitates administration of a single dose of the rotomer pharmaceutical composition. Such devices, which are known in the art, can include a cavity or reservoir where a dry composition and a liquid such as water and/or a non-aqueous solvent may be mixed and then administered to the patient via an opening in the device. Typically, such devices comprise a cavity and a compartment that is separate from the cavity, in which compartment a dry powder can reside. At the time of administration, the powder is released from the compartment into the cavity or reservoir. In some devices, this is accomplished by breaking a barrier that separates the compartment from the cavity or reservoir. Thereafter, the powder may be mixed, typically by shaking, with a liquid in the cavity that may have been added earlier or at the time. The cavity is of sufficient size to hold both the dry rotomer pharmaceutical composition and a quantity of liquid comprising an amount of water and/or non-aqueous solvent sufficient to permit mixing of the dry rotomer pharmaceutical composition to form a liquid composition. The liquid may be added to the container at the time of packaging to create a self-contained product comprising both dry composition and liquid that may be mixed together at the time of administration. Alternatively, the container can contain only a dry rotomer pharmaceutical composition and the liquid is then added prior to administration. The liquid may contain flavoring additives as discussed below. Alternatively, other types of packaging that separate the dry and liquid ingredients may be used. For example, the powder and the liquid can be sealed in 2 form-fill-and-seal pouches, either side by side or one on top of the other and separated by a rupturable seal. The person administering the drug would then rupture the seal and mix the contents back and forth between the 2 compartments until dissolved or suspended.

Once the composition is substantially homogeneous (e.g., from the shaking), it is then administered to the patient via an opening in the device created, e.g., by uncoupling a portion of the device to expose the cavity containing the liquid mixture. For example, a portion of the device, e.g., the top, can be removed by unscrewing a threaded portion from another threaded portion of the container to expose the cavity containing the liquid mixture, which then may be administered to the patient or by the patient. Examples of such devices are provided in U.S. Pat. No. 6,148,996, U.S. application 20080202949, and U.S. Pat. No. 3,156,369. Such single-use devices can be employed for orally administering liquid compositions described herein, especially for prophylaxis or treatment of oral mucositis or its symptoms as described below.

The disclosure thus also provides a kit comprising (i) a rotomer pharmaceutical composition as described above, (ii) a device for oral administration of such compositions. The kit optionally further contains instructions for use.

For such devices, the rotomer pharmaceutical composition may be in a dry form. In such instances, the dry composition which can be present together, e.g., in a compartment as described above, is admixed with water and/or other liquid solvent prior to administration (e.g., by exposing the dry composition to the liquid and shaking) as discussed above.

### Methods of Treating

In certain aspects, the rotomer pharmaceutical compositions and devices described above may be used for treating a human or non-human animal patient in need. The patient typically will be a human patient, although the rotomer pharmaceutical compositions of this disclosure can be used for treating non-human animals, e.g., for veterinary uses. The compositions of this disclosure may be used for preventing or treating a wide variety of diseases and conditions, including diseases and conditions for which treatment with oltipraz is known. Examples of such diseases and conditions include mucositis, HIV, cancers, hepatitis (including HBV and HCV), keratin-based skin diseases, including skin blistering and epidermolysis bullosa simplex and related diseases, dermatitis, inflammatory disorder or disease (including endothelial dysfunction and cardiovascular disease), cachexia, weight loss, sepsis, contrast-induced nephropathy, diabetes. obesity. PCOS, steatosis, hyperlipidemia, and hypertension, chronic kidney disease, pulmonary fibrosis, hypoxic conditions, chemical-induced lung injury, respiratory distress disorder, anon gap acidosis, nephritis, lupus, interstitial lung disease, graft dysfunction, hepatitis, acute kidney injury, noise-induced hearing injuries, poison ingestion, retinopathy, neurotoxicity, cancer-induced injury such as ototoxicity, respiratory infections, autism, conditions involving vasospasm, and conditions considered treatable by provision of n-acetylcysteine, injectable reduced glutathione, or a known intracellular glutathione enhancing agent.

The rotomer pharmaceutical compositions and devices described above may be used for neurological diseases or conditions where a neuroprotective effect is desired, e.g., to prevent, lessen or reduce the effects and/or symptoms associated with stroke injury and ischemia/reperfusion injury. The rotomer pharmaceutical compositions and devices described also above may be used for cardiac diseases or conditions where a cardioprotective effect is desired, e.g., to prevent, lessen or reduce the effects and/or symptoms associated with cardiac ischemia caused by reduced blood flow and oxygen to the heart muscle, leading to or resulting from myocardial infarction and heart tissue death.

The rotomer pharmaceutical compositions and devices described above also may be used to treat an individual who has a viral infection in order to lessen, reduce or eliminate the effects and/or symptoms associated with the viral infection. Such viral infections can include retroviral infections, SARS (severe acute respiratory syndrome) coronavirus and other coronavirus infections. Alternatively, or in addition, the rotomer pharmaceutical compositions and devices described above also may be used to treat an individual who is at risk of a viral infection in order to prepare the individual to better withstand a viral infection if it occurs. Such “host hardening” in advance of a viral infection can lessen, reduce or eliminate some or all of the effects and/or symptoms experienced by the individual in the event that he/she experiences a viral infection, which can be especially beneficial for persons at risk from viral infections such as retroviral infections, SARS (severe acute respiratory syndrome) coronavirus and other coronavirus infections, e.g., individuals having hypertension, decreased pulmonary function, or respiratory diseases or conditions such as pneumonia.

Typically, the rotomer pharmaceutical composition is provided to the patient in an effective amount. The term “effective amount” is used herein to refer to an amount of the rotomer pharmaceutical composition sufficient to produce a significant biological response (e.g., a significant decrease in inflammation). Actual dosage levels of the rotomer pharmaceutical compositions can be varied so as to administer an amount that is effective to achieve the desired therapeutic response for a particular subject and/or application. Of course, the effective amount in any particular case will depend upon a variety of factors including formulation, route of administration, combination with other drugs or treatments, severity of the condition being treated, and the physical condition and prior medical history of the subject being treated.

As used herein, the term “subject” includes both human and animal subjects, and thus veterinary therapeutic uses are provided in accordance with this disclosure. The terms “treatment” or “treating” relate to any treatment of a condition of interest (e.g., mucositis, an inflammatory disorder or a cancer), including but not limited to prophylactic treatment and therapeutic treatment. As such, the terms “treatment” or “treating” include, but are not limited to: preventing a condition of interest or the development of a condition of interest: inhibiting the progression of a condition of interest; arresting or preventing the further development of a condition of interest; reducing the severity of a condition of interest; ameliorating or relieving symptoms associated with a condition of interest; and causing a partial, substantially complete, or complete regression of a condition of interest or one or more of the symptoms associated with a condition of interest.

The rotomer pharmaceutical compositions and devices described above are suitable for treating patients who are suffering from mucositis or who will undergo a treatment such as radiation treatment or chemotherapy that can lead to mucositis, e.g., in the oral cavity (including in the buccal cavity), in the alimentary canal, in the colon and/or rectum, and/or on the skin. Where the mucositis is oral mucositis, the compositions and devices described above for oral delivery may be preferred. Such patients, e.g., may be undergoing, or about to undergo chemotherapy and/or radiation therapy, e.g., radiation treatment in the head and neck area, or to another area of the body.

The rotomer pharmaceutical compositions and devices above may be used to accomplish one, more than one, or all of the following beneficial effects on human or non-human animal patients, i.e., (i) prophylactically prevent or delay the onset of mucositis, including oral mucositis (e.g., inflammation of the mucosa), (ii) treat existing mucositis, including oral mucositis (iii) alleviate symptoms associated with mucositis, including oral mucositis (iv) reduce or lessen the severity of existing mucositis, including oral mucositis (v) hasten the cure or healing of mucositis, including oral mucositis (vi) reduce the incidence and/or duration of mucositis, including oral mucositis, e.g.. mild, moderate and severe oral mucositis, (vii) prophylactically prevent or delay the onset of weight loss or cachexia by a patient with oral mucositis, (viii) reduce the amount of weight loss or cachexia experienced by a patient with oral mucositis, and/or (ix) increase the ability of a patient with oral mucositis to take food by mouth. Such compositions also may be used for the prevention and/or treatment of patients with dysphagia (difficulty swallowing), e.g., cancer patients, or to delay the onset of dysphagia or lessen the severity of dysphagia, e.g., in cancer patients. Such compositions also may be used for the prevention and/or treatment of patients with xerostomia (the subjective feeling of oral dryness), or to delay the onset of xerostomia, lessen the severity of xerostomia, and/or reduce the incidence of moderate-to-severe xerostomia. In certain aspects, the single-use devices described above may be used for administration of liquid compositions for accomplishing one, more than one, or all of the above relating to oral mucositis, dysphagia and xerostomia. Advantageously, formulations are also non-irritating, well-tolerated, palatable (if orally administered), non-cytotoxic, weakly or non-sensitizing. non-sensitizing.

Certain aspects herein provide methods for treating mucositis, comprising administering to a patient in need thereof a therapeutically effective amount of a rotomer pharmaceutical composition as described herein. The disclosure also provides a rotomer pharmaceutical composition as described herein for use in the treatment of mucositis. The disclosure also provides the use of a rotomer pharmaceutical composition as described herein in the manufacture of a medicament for the treatment of mucositis. The administration of the formulation to a patient may be an oral administration, including buccal administration. The methods of administration described herein can represent a treatment regimen of a predetermined duration. e.g., 1 month. 2 months, 3 months, 4 months, 5 months, 6 months, or longer. Rotomer pharmaceutical compositions according to this disclosure can be applied or administered once daily, twice daily, three times daily, or as needed. In situations where the patient is undergoing chemotherapy and or radiation therapy, the dosage may be administered prior to a treatment, e.g., within 1 hour, within 3 hours, within 6 hours, within 12 hours, within 24 hours, or more than 24 hours before the treatment. For example, dosing can begin several days in advance of receiving chemotherapy, radiation therapy and/or chemoradiation therapy. For example, the patient can begin receiving treatment once per day, or more than once per day, beginning three days in advance of starting therapy, and then receive dosing each day during the course treatment, including days when therapy is not received. On days when treatment is being given, e.g., dosing is given prior to therapy, e.g., within an hour before therapy is administered. Dosing can be given anytime during the day, e.g., in the morning, on days when therapy is not received. Additionally, or alternatively, the dosage may be administered after a treatment, e.g., within 1 hour, within 3 hours, within 6 hours, within 12 hours, within 24 hours after the treatment, or more than 24 hours after the treatment.

This disclosure thus provides methods for treating mucositis comprising administering to a patient in need thereof a therapeutically effective amount of a rotomer pharmaceutical composition described above. The disclosure also provides rotomer pharmaceutical compositions as described above for use in the treatment of mucositis. The disclosure also provides the use of a rotomer pharmaceutical composition as described herein in the manufacture of a medicament for the treatment of mucositis. The administration of the rotomer pharmaceutical composition to a patient may be an oral administration, including buccal administration.

Where liquid compositions are administered, the composition may be administered orally or parenterally, e.g., by subcutaneous, intramuscular, intrastemal, or intravenous injection. Where oral administration is employed, the liquid composition simply may be swallowed, or it may be administered by a “swish and swallow” regimen or a “swish and spit” regimen. By administering the composition orally in a liquid form to a patient who has oral mucositis or who is at risk of developing oral mucositis (e.g., undergoing or about to undergo chemotherapy, radiation therapy and/or chem-radiation therapy), the compositions may provide a therapeutic benefit in terms of the mucositis as described above, i.e., it may prophylactically prevent or delay the onset of mucositis, treat existing mucositis, alleviate or decrease symptoms associated with mucositis (e.g., inflammation and bleeding of the mucosa), reduce or lessen the severity of existing mucositis, reduce the duration of the mucositis, and/or hasten the reduction, cure or healing of mucositis. In such cases, liquid compositions comprising an ingredient with a negative charge, e.g., a cationic surfactant or polymer such as Eudragit RL, may provide a further advantage by virtue of providing an adherence or association with the mucosa of the mouth, which tends to have a positive charge. The physical and chemical properties of aspects of the compositions described herein can impart characteristics to the formulation such as stability, delivery of the active agent to the mucosal membrane, and ease of administration.

As noted above, the rotomer pharmaceutical compositions as described herein may be co-administered with other therapeutic agents, either together or separately as part of a therapeutic regimen. Such agents include N acetylcysteine and/or other antioxidants, pantothenic acid (vitamin B5) or other agents that enhance glutathione synthesis, glutathione, e.g., for topical administration, Medihoney (for topical administration), curcumin (for topical administration) or other NF-kappaB inhibitors, Mesalamine and/or other anti-inflammatory agents, e.g., for oral or rectal administration compositions, and superoxide dismutase or other compounds that prevent damage from reactive O₂⁻ (superoxide).

### EXAMPLES

Certain aspects of this disclosure are further illustrated by the following examples, which should not be construed as limiting the scope of the appended claims in any way.

### Example 1

### Method to Make the Hydrochloride of Oltipraz: (+) Rotomer

20 mg of synthesized oltipraz powder (ST-617-API, discussed below) is recrystallized using acetonitrile:water and added into a 5 ml glass vial equipped with a rubber septum. To the vial is added 1 ml of dry chloroform at room temperature and the vial set on a vibrating table until the solution is clear. Using a gas syringe, 5 ml of anhydrous hydrochloric acid gas is slowly bubbled into the solution over 5 minutes with intermittent shaking of the vial. Once all the HCl gas has been added, the vial is shaken for 5 minutes, the septum is removed, and the solvent gently evaporated under a slow steady stream of nitrogen gas to give the hydrochloride complex of oltipraz as an orange/red solid. FTIR: C=S peaks 1203/1211 (relative intensity 3:7) and 433/421 (relative intensity 2:8).

### Example 2

### Method to Make Hydroxymaleate Anhydride Complex of Oltipraz: (−)-Rotomer

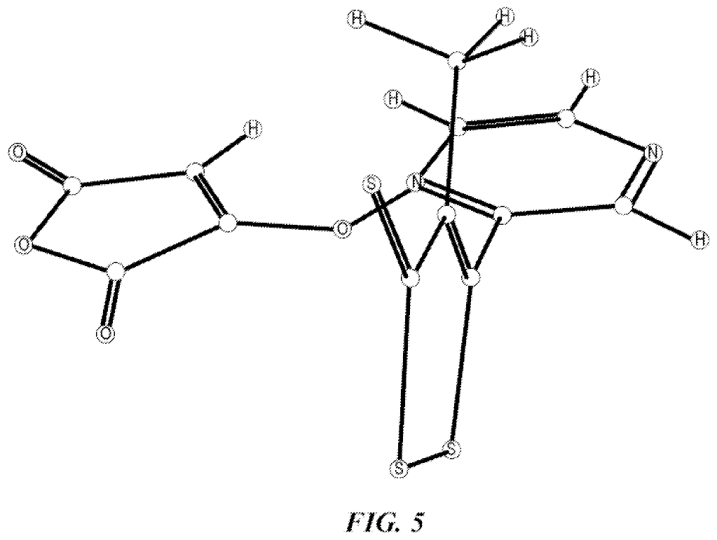

20 mg of synthesized oltipraz powder is recrystallized using acetonitrile:water and added into a 10 ml glass vial equipped with a rubber septum. All moisture is removed from the vial by nitrogen gas using a gas syringe. The vial is placed in an ice-bath and into it is added a pre-chilled (10° C.) solution of 22 mg of (+)-diacetyl-L-tartaric anhydride in 5 ml of ultra-dry acetone, shaken on a vibrating table for 5 minutes, and after 5 minutes is added 0.5 ml of glacial acetic acid over 30 seconds, using a syringe. After 4 h at 0-10° C. with intermittent shaking, the vial is allowed to come to room temperature. The septum is removed, and the solvent gently evaporated under a slow steady stream of nitrogen gas to give the hydroxymaleate anhydride complex of oltipraz (see FIG. 5) as orange solid. The complex is washed 3 times with ethyl ether to remove traces of acetic acid. FUR: C=S peaks 1201/1214 (relative intensity 8:2) and 434/423 (relative intensity 8:2). A 2 mg sample of the oltipraz-complex is ground with KBR and made into a standard pellet for FTIR analysis on a Perkin Elmer 1600 series FTIR.

### Example 3

### Method to Make the Acetyl Chloride Complex of Oltipraz: (+) Rotomer

20 mg of synthesized oltipraz powder is recrystallized using acetone:water and added into a glass vial equipped with a rubber septum. To the vial was added 10 ml of dry chloroform at room temperature and the vial set on a vibrating table until the solution is clear. Using a syringe. 5 ml of anhydrous acetyl. chloride is slowly added into the solution with intermittent shaking of the vial. Once all the acetyl chloride has been added, the vial is shaken for 15 minutes, the septum is removed, and the solvent and excess reagent gently evaporated under a slow steady stream of nitrogen gas to give the acetyl chloride complex of oltipraz as an orange solid. FTIR: C=S peaks 1202/1212 (relative intensity 3:7) and 432/422 (relative intensity 2:8).

### Example 4

### Method to Make the N-Butyl Chloride Complex of Oltipraz: (+) Rotomer

20 mg of synthesized oltipraz powder is recrystallized using acetone:water and added into a glass vial equipped with a rubber septum. To the vial was added 10 ml of dry chloroform at room temperature and the vial set on a vibrating table until the resulting solution is clear. Using a syringe, 5 ml of n-butyl chloride is slowly added into the solution with intermittent shaking of the vial. Once all the n-butyl chloride has been added, the vial is warmed to 65° C. and shaken for 30 minutes, the septum is removed, and the solvent and excess reagent gently evaporated under a slow steady stream of nitrogen gas to give the n-butyl chloride complex of oltipraz as a red solid. FTIR: C=S peaks 1201/1213 (relative intensity 2:8) and 433/421 (relative intensity 2:8).

### Example 5

### Method to Make the Methyl Iodide Complex of Oltipraz: (+) Rotomer

50 mg of synthesized oltipraz powder is recrystallized using acetone:water and added into a pressure-rated glass vial equipped with a screw seal. To the vial was added 20 ml of dry chloroform at room temperature and the vial set on a vibrating table until the resulting solution is clear. Using a syringe, 10 ml of methyl iodide is slowly added into the solution with intermittent shaking of the vial. Once all the methyl iodide has been added, the vial is sealed and heated to 120° C. under pressure and shaken for 3 hours (“hrs”), the vial is cooled and unsealed, and the solvent and excess reagent gently evaporated under a slow steady stream of nitrogen gas to give the methyl iodide complex of oltipraz as a red/brown solid. FT1R: C=S peaks 1197/1214 (relative intensity 2:8) and 431/419 (relative intensity 2:8).

### Example 6

### Method to Make the Benzoyl Chloride Complex of Oltipraz: (+) Rotomer

20 mg of crude oltipraz powder is added into a glass vial equipped with a rubber septum. To the vial was added 10 ml of dry chloroform at room temperature and the vial set on a vibrating table until the resulting solution is clear. Using a syringe, 1 ml of anhydrous benzoyl chloride is slowly added into the solution with intermittent shaking of the vial. Once all the benzoyl chloride has been added, the vial is shaken for 15 minutes, the septum is removed, and the solvent and excess reagent gently evaporated under 10 mm Hg vacuum to give the benzoyl chloride complex of oltipraz as a red solid. FTIR: C=S peaks 1200/1213 (relative intensity 3:7) and 431/419 (relative intensity 3:7).

### Example 7

### Method to Make the Diethyl Dithiophosphate Complex of Oltipraz: (−) Rotomer

22 mg of synthesized oltipraz powder is recrystallized using acetone:water and added into a glass round bottom flask equipped with a magnetic stirrer and cold-water condenser. To the flask was added 10 ml of acetone at room temperature and 20 mg of diethyl dithiophosphate ammonium salt is slowly added into the solution while stirring is continued. Once all the diethyl dithiophosphate has been added, the temperature is increased and set to reflux for 3 hrs. Released ammonia is captured in a dilute hydrochloric acid trap from the top of the condenser. After 3 hrs the solvent is removed to yield the diethyl dithiophosphate complex of oltipraz as an orange/red solid. FTIR: C=S peaks 1201/1220 (relative intensity 9:1) and 472/444 (relative intensity 8:2)

### Example 8

### Method to Make the Diethyl Thiophosphate Complex of Oltipraz: (−) Rotomer

22 mg of synthesized oltipraz powder is recrystallized using acetone:water and added into a glass round bottom flask equipped with a magnetic stirrer and cold-water condenser. To the flask was added 10 ml of acetone at room temperature and 19 mg of diethyl thiophosphate ammonium salt is slowly added into the solution while stirring is continued. Once all the diethyl thiophosphate has been added, the temperature is increased and set to reflux for 3 hrs. Released ammonia is captured in a dilute hydrochloric acid trap from the top of the condenser. After 3 hrs the solvent is removed to yield the diethyl dithiophosphate complex of oltipraz as an orange/red solid. FTIR: C=S peaks 1208/1221 (relative intensity 9:1) and 4791442 (relative intensity 9:1).

### Example 9

### Method to Make the Dimethyl Phosphate Complex of Oltipraz: (−) Rotomer

75 mg of trimethyl phosphate was placed with 100 mg of crude oltipraz (95% purity), in a round bottom flask equipped with a condenser and stirrer and heated at 80° C. for 72 hr. After 72 hrs, the mixture is cooled to room temperature and washed with 50 ml of toluene five times. 255 g of orange/red dimethyl phosphate complex of N-methyl oltipraz is collected from the lower layer and dried under vacuum at 60° C. FTIR: C=S peaks 1210/1227 (relative intensity 9:1) and 489/453 (relative intensity 9:1) .

### Example 10

### Method to Make the Isethionic Acid Complex of Oltipraz: (−) Rotomer

22 mg of crude oltipraz powder is added into a glass round bottom flask equipped with a magnetic stirrer and cold-water condenser. To the flask was added 10 ml of acetone at room temperature and 30 mg of isethionic acid ammonium salt is slowly added into the solution while stirring is continued. Once all the isethionic acid ammonium salt has been added, the temperature is increased and set to reflux for 6 hrs. Released ammonia is captured in a dilute hydrochloric acid trap from the top of the condenser. After 6 hrs the reaction is brought to pH 6.8 with minimum quantity of concentrated hydrochloric acid and the resultant mixture is evaporated, under vacuum, to dryness to yield the isethionic acid complex of oltipraz as a red/brown solid. FTIR: C=S peaks 1216/1228 (relative intensity 9:1) and 464/435 (relative intensity 9:1).

### Example 11

### Method to Make the Thiobenzoic Acid Complex of Oltipraz: (−) Rotomer

20 mg of crude oltipraz powder is added into a glass vial equipped with a rubber septum. To the vial was added 10 ml of dry chloroform at room temperature and the vial set on a vibrating table until the resulting solution is clear. Using a syringe, 1 ml of thiobenzoic acid ammonium salt is slowly added into the solution with intermittent shaking of the vial. Once all the thiobenzoic acid ammonium salt has been added, the temperature is increased and set to reflux for 1 hr. The released ammonia is captured in a dilute hydrochloric acid trap from the top of the condenser. After 1 hour the contents are removed under vacuum to yield the thiobenzoic acid complex of oltipraz as an orange solid. FTIR: C=S peaks 1210/1223 (relative intensity 9:1) and 474/438 (relative intensity 9:1)

### Example 12

### Effect of (+) and (−) Rotomers on ROS Production in H₂O₂-Induced Oxidatively Stressed HGEPp Cells

The formation of reactive oxygen species (ROS) is indicative of oxidative stress.

Accumulation of ROS coupled with an increase in oxidative stress is implicated in the pathogenesis of many diseases, one of which is mucositis. See Sonis, A biological approach to mucositis, J Support Oncol 2004;2:21-36; Halliwell & Whiteman. Measuring reactive species and oxidative damage in vivo and in cell culture: how should you do it and what do the results mean?, British J. Pharmacology, Volume 142, Issue 2. May 2004. 231-255; and Iglesias-Bartolome et al., mTOR Inhibition Prevents Epithelial Stem Cell Senescence and Protects from Radiation-Induced Mucositis, Cell Stem Cell 11, 401-414, Sep. 7, 2012). Free radicals and other reactive species are constantly generated in vivo and cause oxidative damage to biomolecules, a process held in check by multiple antioxidant and repair systems.

An assay to determine the effect of oltipraz and formulated oltipraz crystals on intracellular ROS using HGEPp cells is described in Example 7 of PCT/IB2017/001312, published as WO 2018/047013 to Applicant ST IP Holding AG. The formulated oltipraz crystals were prepared generally according to the process described in Example 1 of ACT/1132017/001312 (MHD less than about 350 nm). That same assay was carried out on the rotomer compositions prepared in Examples 1-11 above to determine their effect on protecting primary human gingival epithelial cells (HGEPp) cells from oxidative damage induced by hydrogen peroxide (H₂O₂).

### Conditions:

| | |
|---|---|
| Normal | normal control cells were cultured in DPBS |
| | containing 0.3% DMSO. |
| Positive control (PC) | oxidative stressed cells after treatment with 0.3 |
| | mM H₂O₂ for 4 h. |
| Formulated oltipraz | group of cells pretreated for 24 hrs with |
| crystals (ST-617-DPI) | ST-617-DPI at 100 μg/mL prior to treatment |
| | with H₂O₂. |
| (+) rotomer complexes | group of cells pretreated for 24 hrs with (+) |
| | rotomer complex at 100 μg/mL prior to treatment |
| | with H₂O₂. |
| (−) rotomer complexes | group of cells pretreated for 24 hrs with (−) |
| | rotomer complex at 100 μg/mL prior to treatment |
| | with H₂O₂ |

### Results:

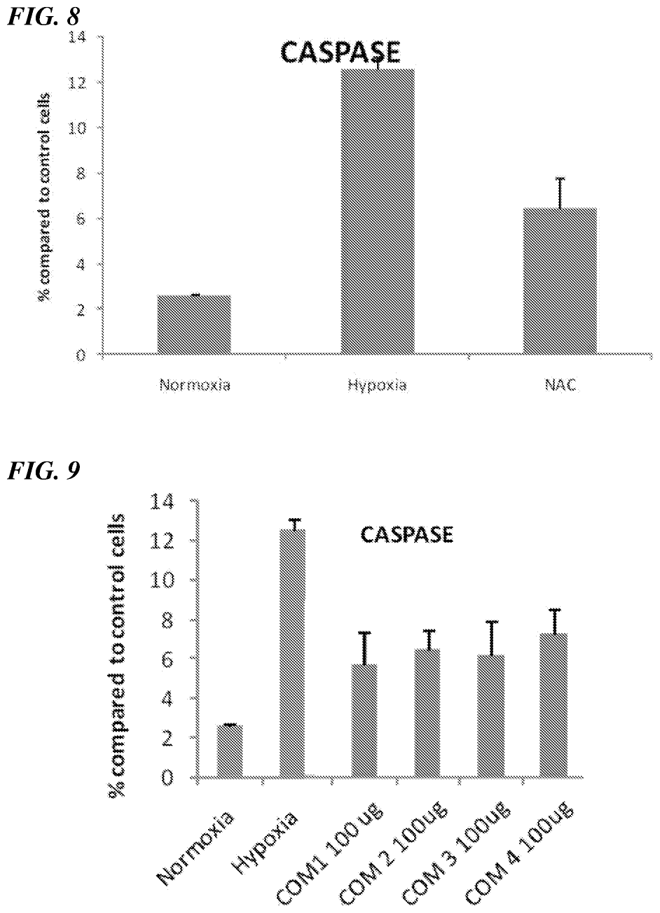

The results of the above assay on ST-617-DPI and the rotomer complexes of Examples 1-11 are shown below in Table 1. The result for ST-617-API (recrystallized oltipraz prepared according to the process disclosed in WO2016207914) reported in Table 1 below is taken from FIG. 9 of PCT/IB20171001312 (100 μg/mL pretreatment).

TABLE 1

| | 100% |
|---|---|
| Normal | (optical density baseline) |
| Positive control (PC) | 139% |
| Recrystallized oltipraz (ST-617-API) | 124% |
| Formulated oltipraz crystals (ST-617-DPI) | 122% |
| Example 1 | 128% |
| Example 2 | 116% |
| Example 3 | 129% |
| Example 4 | 128% |
| Example 5 | 134% |
| Example 6 | 131% |
| Example 7 | 117% |
| Example 8 | 115% |
| Example 9 | 112% |
| Example 10 | 113% |
| Example 11 | 116% |

The reduction in ROS levels for the group of cells treated with 100 μg/mL of ST-617-DPI in the assay (122%) as compared to the control (139%) were consistent with the results reported in PCT/IB2017/001312 for 100 μg/mL (123%), and with other results obtained for the ST-617-DPI in this assay. The (+) rotomer complexes provided a modest decrease in ROS whereas the (−) rotomer complexes provided a substantial decrease. The assay results for the (−) rotomer were statistically significant at a 90% confidence level,

### Example 13

### Differentially Regulated Oxidative Stress-Related Genes in HGEPp Cells Following Treatment with ST-617-DPL ST-617-API and the Rotomer Complexes of Examples 1-11

The Nrf2 system (discussed above) is considered to be a major cellular defense mechanism against oxidative damage by activating genes that encode phase H detoxifying and antioxidant enzymes. One such oxidative damage is oral mucositis which occurs during and after chemotherapy treatment particularly in head and neck cancer treatments. The Human oxidative stress PCR array was used to evaluate the relative expression of 84 stress genes after pretreating with 100 μg/ml of formulated oltipraz composition prepared generally in accordance with the process described in Example 1 of PCT/1B2017/001312 (MHD less than about 350 nm), the rotomeric complexes described in Examples 1-11 above, and negative control (formulated oltipraz composition without the oltipraz) within HGEPp cells.

### Materials

HGEPp cells were purchased from CellnTec Advanced Cell Systems AG. RNEasy Plus Micro Kit was purchased from Qiagen N.V., USA. RNase-free DNase Set was purchased from Qiagen N.V., USA. RT2 Easy First Strand Kit (DNA generator) and RT2 SYBR® Green fluor qPCR mastermix were purchased from Qiagen N.V., USA. Oxidative Stress RT2 profiler PCR arrays (84 protective genes) were purchased from Qiagen N.V., USA. The iCycler PCR system from Bio-Rad Inc., USA was used for the RT-PCR.

### Methods

### Cell Culture:

Pooled primary HGEPp cells were propagated in CnT-Prime epithelial culture medium provided by CellnTec on 100 mm petri dishes coated with 30 mg/ml Type I rat tail collagen (BD Biosciences) diluted in Duibecco's phosphate-buffered saline (DPBS). HGEPp cells were grown in 12×60 mm Nunc™ Cell Culture dishes at a 2.5×104 cells/cm2 density concentration.

### Preparation of the Dosing Solutions:

Formulated oltipraz crystals (ST-617-DPI) were prepared generally according to the process described in Example 1 of PCT/IB2017/001312. Recrystallized oltipraz was prepared according to the process disclosed in WO2016207914 ST-617-API. The ST-617-DPI. ST-617-API and rotomeric complexes of Examples 1-11 were dissolved in a DMSO to arrive at a final concentration of 100 μg/ml. All dosing solutions contained 0.3% of DMSO which is well below the maximum tolerated DMSO percent of 0.8% for HGEPp cells.

Pre-Treatment of HGEPp cells with ST-617-DPI and rotomer complex solutions:

1. Dish cell concentration was selected to be 2.5×105/ml to yield an OD absorbance within the linear portion of the control curve.

2. Once the HGEPp cells were cultured and ready (had reached confluence) on the dishes, the media was removed and discarded. The cells were washed gently with DPBS 2-3 times and the last wash removed and discarded.

3. The HGEPp cell culture dishes were then pretreated for 24 h with ST-617-API, ST-617-DPI and the rolomeric complexes at 100 μg/mL concentrations (in duplicate) at 37° C.

mRNA Preparation from HGEPp Cells:

The RN-easy UCP Micro Kit was used to purify mRNA from the HGEPp cells.

1. After discarding and washing treatments, the cells were pelleted by centrifuging for 5 min at 1000 RPM in a centrifuge tube. All the supernatant was carefully removed by aspiration, making sure all the cell medium has been removed thoroughly.

2. The cells were disrupted by adding 350 μl buffer RULT taking care to loosen the cell pellet from the tube and vortexed to mix thoroughly and the mixture was homogenized by passing the lysate 5 times through 20-gauge needle fitted to an RNase-free syringe.

3. Added 350 μl of 70% ethanol to the lysate and it was mixed again by pipetting. (Some precipitate is visible but is not a problem for the assay)

4. Transferred the sample, including any precipitate that may have formed, to an RNeasy UCP MinElute spin column placed in a 2 ml collection tube and centrifuged for 15 s at 10,000 rpm. Discarded the flow through.

5. Added 3500 Buffer RUWT to the spin column and centrifuged for 15 s at 10,000 rpm to wash the membrane. Discarded the flow thru.

6. Added 10 μl DNase I stock solution to 70 μl Buffered RDD. Mixed gently, added 80 μl of the DNase i mix directly to the spin-column membrane and vortexed for 15 min.

7. Added 35011 Buffer RUWT to the spin column and centrifuged for 15 s at 10,000 rpm to wash it and discarded the flow thru.

8. Placed the spin column in a new 2 ml collection tube, add 500 μl Buffer RUPE and centrifuged for 15 s at 10,000 rpm. Discarded the flow thru.

9. Washed the spin column again with 500 μl of 80% ethanol and placed it in a new 2 ml collection tube and centrifuge for 5 mins at full speed with the lid open to make sure all ethanol is removed.

10. Placed the spin column in a new 1.5 ml collection tube, added 14 μl ultra-clean water directly to the center of the spin-column membrane, closed the lid and centrifuged for 1 min at full speed to elute the RNA. The dead volume of the spin column is 4 μl. Eluted with 16 μl ultra-clean water to yield a 20 μl (4 μg) RNA eluate.

cDNA Prep for RT-PCR:

1. A RNA sample from each of the above treatments was added to 40 μl of Buffer GE2 (gDNA elimination buffer) and RNase-free H₂O to make a final volume of 60

2. Incubated at 37° C. for 5 min and immediately placed on ice for 2 minutes.

3. Added 62 μl of the BC5 Reverse Transcriptase Mix to each 60 μl RNA sample for a final volume of 102 μl.

4. Incubated at 42° C. for exactly 1.5 minutes and then immediately stopped the reaction by heating at 95° C. for 5 minutes. (held on ice until qPCR, if required).

### Human Oxidative Stress RT2 Profiler PCR Array:

RT-PCR is a highly sensitive and reliable method for gene expression analysis. The assay was reliably used to analyze expression levels of 84 genes related to oxidative stress in HGEPp cells pre-treated with ST-617-APT, ST-617-DPT and the rotomeric complexes at 100 μg/ml for 24 h. The cDNA from above was mixed with the RT2 SYBR Green fluor Mastermix and aliquoted into the wells of the RT2 Profiler PCR Array. RT-PCR was performed on an iCycler. Gene expression was compared using et values and the results were calculated using ΔΔ Ct method with normalization to the average expression levels of the five common genes (ACTB, B2M, GAPDH, HPRT, and RPL13A).

### Results:

The results of the assays on the ST-617-DPI and rotomer complexes of Examples 1-11 are shown below in Table 2. The results for ST-617-AP1 (recrystallized oltipraz prepared according to the process disclosed in WO2016207914) reported in Table 2 below are from an earlier assay conducted as described above.

TABLE 2

| | SAMPLE | | | | | | | | | | | | | | |
|---|---|---|---|---|---|---|---|---|---|---|---|---|---|---|---|
| | | | | | | | | | | | | | | (+) | (−) |
| | ST-617 | ST-617 | Ex. | Ex. | Ex. | Ex. | Ex. | Ex. | Ex. | Ex. | Ex. | Ex. | Ex. | rotomer | rotomer |
| GENE | API | DPI | 1 | 2 | 3 | 4 | 5 | 6 | 7 | 8 | 9 | 10 | 11 | avenge | average |
| | UP REGULATION RELATIVE FOLD CHANGE | | | | | | | | | | | | | | |
| ALOX12 | 6.15 | 6.42 | 6.38 | 6.46 | 6.33 | 5.82 | 6.04 | 5.93 | 6.38 | 6.41 | 6.43 | 6.48 | 6.39 | 6.07 | 6.43 |
| SOD1 | 2.46 | 4.88 | 3.86 | 5.13 | 4.24 | 4.15 | 3.89 | 3.92 | 4.85 | 4.86 | 5.22 | 5.23 | 4.96 | 4.04 | 5.04 |
| GPX1 | 4.88 | 4.82 | 4.81 | 4.8 | 4.76 | 4.80 | 4.98 | 4.43 | 4.72 | 4.81 | 4.89 | 4.86 | 4.82 | 4.77 | 4.82 |
| NQO1 | 2.95 | 3.67 | 3.14 | 4.07 | 3.36 | 3.34 | 3.15 | 3.17 | 3.93 | 3.92 | 4.24 | 4.26 | 4.09 | 3.24 | 4.09 |
| MPO | | 3.35 | 3.22 | 3.21 | 3.10 | 3.20 | 3.18 | 3.05 | 3.42 | 3.47 | 3.41 | 3.39 | 3.44 | 3.15 | 3.39 |
| GCLC | 4.14 | 3.26 | 3.35 | 4.52 | 3.27 | 3.35 | 3.38 | 3.31 | 4.41 | 4.39 | 4.60 | 4.52 | 4.46 | 3.33 | 4.48 |
| GPX4 | | 2.91 | 2.86 | 2.84 | 2.83 | 2.96 | 2.85 | 2.91 | 2.99 | 3.02 | 2.93 | 2.79 | 2.94 | 2.86 | 2.92 |
| GSTP1 | 2.17 | 2.98 | 2.77 | 3.07 | 2.87 | 2.88 | 2.90 | 2.74 | 3.13 | 3.08 | 3.16 | 3.18 | 3.07 | 2.83 | 3.12 |
| GCLM | 3.03 | 2.19 | 2.33 | 3.01 | 2.38 | 2.38 | 2.27 | 2.25 | 3.00 | 3.02 | 3.15 | 3.11 | 3.08 | 2.32 | 3.06 |
| | DOWN REGULATION RELATIVE FOLD CHANGE | | | | | | | | | | | | | | |
| GTF2i | 0.34 | 0.32 | 0.33 | 0.26 | 0.32 | 0.35 | 0.33 | 0.36 | 0.29 | 0.29 | 0.28 | 0.31 | 0.29 | 0.34 | 0.29 |
| PRDX2 | | 0.29 | 0.31 | 0.26 | 0.33 | 0.32 | 0.32 | 0.31 | 0.28 | 0.28 | 0.27 | 0.28 | 0.27 | 0.32 | 0.27 |
| PTGS1 | 6.22 | 0.24 | 0.28 | 0.23 | 0.29 | 0.27 | 0.28 | 0.28 | 0.21 | 0.23 | 0.21 | 0.22 | 0.22 | 0.28 | 0.22 |
| UCP2 | 0.18 | 6.20 | 0.26 | 0.15 | 0.26 | 0.25 | 0.29 | 0.28 | 0.17 | 0.14 | 0.15 | 0.16 | 0.15 | 0.27 | 0.15 |

The negative control showed no change in any gene regulation. All the rotomers showed higher gene regulating responses for the (−) rotomer over the (+) rotomer. The highest-fold changes between two rotomers were for SOD1, GCLC, GCLM and UCP2. It is noteworthy that GCLC and GCLM genes, which are glutathione (GSH) biosynthesis genes, showed significant consistent differences between the (+) and (−) rotomer. These results correlate with increases in serum GSH observed in patients in a clinical trial who received ST-617-DPI and experienced low oral mucositis development while undergoing radiation therapy for head and neck cancer. The (−) rotomers showed higher gene upregulating responses compared to ST-617-API for ALOX12, SOD1, NQ01, GCLC and GSTP1. The (−) rotomers showed higher gene upregulating responses compared to ST-617-DPI for SOD1, NQ01, GCLC, GSTP1 and GCLM. Within the (−) rotomers, the dimethyl phosphate and isethionic acid complexes (Examples 9 and 10, respectively) showed significantly higher up-regulation compared to ST-617-DPI and ST-617-DPI for SOD1, NQ01, GCLC, GSTP1 and GCLM. The (−) rotomers also showed a differential down-regulation of UCP2 (Mitochondrial uncoupling protein 2, which is involved in the control of mitochondria-derived ROS) compared to ST-617-DPI and ST-617-API.

The rotomer pharmaceutical compositions and methods of administering the rotomer pharmaceutical compositions of this disclosure thus may be used to treat any human or non-human animal patient to decrease intracellular reactive oxygen species (ROS) and/or decrease oxidative stress, including in patients undergoing treatments that provide oxidative stress such as chemotherapy or radiation therapy.

The rotomer pharmaceutical compositions and methods of administering the rotomer pharmaceutical compositions of this disclosure may be used to treat any human or non-human animal patient to provide an antioxidant effect, including in patients undergoing treatments that provide oxidative stress such as chemotherapy or radiation therapy. The rotomer pharmaceutical compositions and methods of administering the rotomer pharmaceutical compositions of this disclosure also may be used to slow the onset, and/or reduce the severity, and/or reduce the duration of oxidative damage in patients (e.g., mucositis, including oral mucositis), including in patients undergoing treatments that provide oxidative damage such as chemotherapy or radiation therapy.

Based on the above results it is further believed that the (+) rotomer, including compositions in which there is a rotomeric excess of the (+) rotomer, may up-regulate genes associated with Nrf2 pathway. Based on the above results, it is further believed that the (−) rotomer, including compositions in which there is a rotomeric excess of the (−) rotomer, may up-regulate genes associated with Nrf2 pathway.

### Example 14

### Neuroprotective Effect of the Rotomer Complexes of Examples 1, 2, 3 and 9

The effect of the rotomer complexes of Examples 1, 2, 3 and 9 on neurotoxicity induced by oxygen glucose deprivation (OGD) was tested in a caspase 3/7 activation assay. The assay was applied to freshly isolated rat cortical neurons cultured in 96-well-plates and exposed to the compounds at 100 μg/ml.

### Methods

In vitro ischemic injury was induced in rat primary neuronal culture by oxygen-glucose deprivation for 2 hours. Neurons were incubated with the compounds for 1 hour eighteen hours prior to the OGD induction. Dizocilpine (MK801) 10 μM was used as positive neuroprotection control. After treatments, incubated with three optically compatible fluorescent dyes, and subsequently analyzed with BD Pathway 855 (Becton Dickinson). Cell parameters associated with caspase 3/7 activation—indicative of pre-lethal cytotoxic effects—were measured by high content screening at single cell level. As discussed below, each of the rotomer complexes exhibited a high degree of neuroprotection in this assay.

Primary cultures of cortical neurons were isolated from cerebral cortices of Sprague-Dawley rat fetuses at embryonic day 18. Brains were removed and after removing the meninges, the tissues were dissected under a binocular microscope. Neurons were enzymatically dispersed by trypsin 0.2% and DNAse 10.04% digestion for 10 min at 37° C. plated in poly-L-lysine wells and incubated with B27 supplemented neurobasal medium.

Cortical neurons of 18 days old embryonic rats were plated in poly-1-lysine coated 96-well plates (30,000 cells per well). Cells were maintained in neurobasal medium supplemented with B-27 component: for 5 days at 37° C. in a humidified 5% CO2 atmosphere. At day 5, cells were subjected to oxygen glucose deprivation (OGD) for 2 hours at 37° C. The cultures were placed in an aerobic chamber (Billops-Rothenberg) and incubated in neurobasal medium lacking glucose and B27 factor and aerated with an anaerobic gas mixture (94.7% N2. 5% CO2, 0.3% O₂) to remove residual oxygen. Control cultures were kept in the original neurobasal medium but were submitted to the anaerobic conditions. At the end of the OGD conditions, the cells were removed from the anaerobic chamber, the OGD medium was replaced with neurobasal medium containing glucose, and the cells were incubated for an additional 24 h. The test compounds were added to the neurons eighteen hours prior to the OGD induction and maintained for 1 h. Replicate cultures were treated as described above with dizocilpine (MK801) as positive control of neuroprotection. The neuroprotective potential of the four rotomer complexes was determined by a caspase 3/7 activation assay.

Caspase 3/7 activation assay:

Caspase 3/7 activation was determined using The CellEvent® Caspase-3/7 Green Detection Reagent which is intrinsically a non-fluorescent peptide that inhibits the ability of the dye to bind to DNA. However, after caspase-3/7 activation in apoptotic cells, the peptide is cleaved allowing the dye to bind to DNA producing a bright, fluorogenic response. Cell were stained with 5 μM reagent, washed 3 times and measured at 488 nm/530 nm Ex/Em. This dye permits the direct quantification of apoptotic cells.

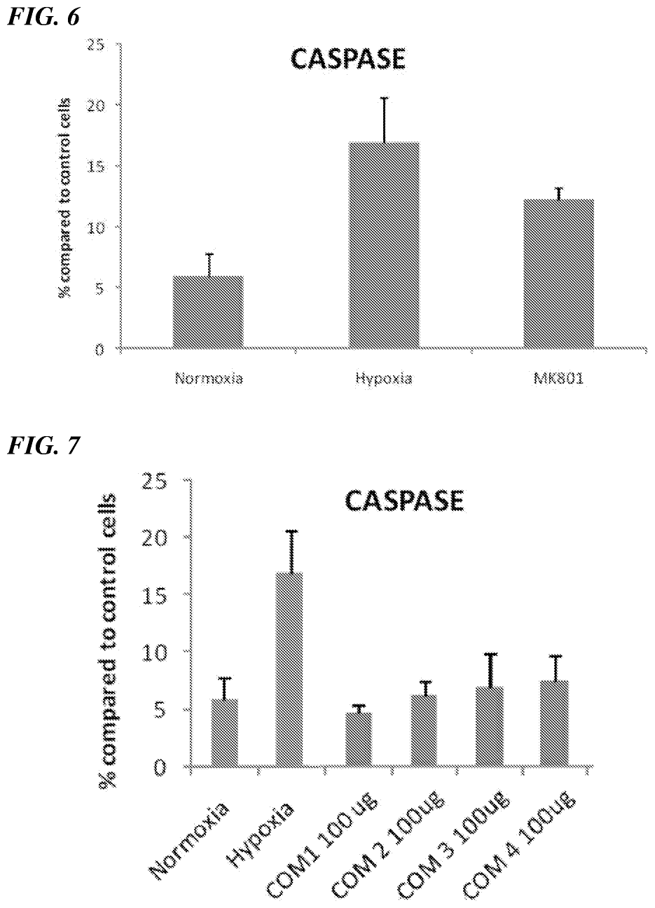

OGD treatment resulted in an increase of three-fold of caspase 3/7 activation in neurons when measured 24 h after exposure whereas the neuroprotection positive control MK801, prevents OGD-induced apoptosis by 28% based on caspase activation. FIG. 6 shows the results of the Caspase 3/7 activation assay of positive and negative controls. Data points represent the mean+SD for each condition of number of apoptotic cells per nuclei.

OGD induced caspase 3/7 activation could be also prevented by the application of the four rotomer complexes (100 μg/ml), which significantly reduced caspase 3/7 activation and improved survival of neurons (FIG. 7). In FIG. 7, COM 1 is the hydroxymaleate anhydride complex of oltipraz: (−)-rotomer described in Example 2 above. COM 2 is the hydrochloride of oltipraz: (+) rotomer described in Example 1 above. COM 3 is the dimethyl phosphate complex of oltipraz: (−) rotomer described in Example 9 above, and COM 4 is the acetyl chloride complex of oltipraz: (+) rotomer described in Example 3 above. Data points represent the mean ±SD for each condition of number of apoptotic cells per nuclei. As shown in FIG. 7, each of the rotomer complexes exhibited a high degree of neuroprotection.

### Example 15

### Cardioprotective Effect of the Rotomer Complexes of Examples 1, 2, 3 and 9

The effect of the rotomer complexes of Examples 1, 2, 3 and 9 on cardiotoxicity induced by oxygen glucose deprivation (OGD) was tested in a caspase 3/7 activation assay. The assay was applied to “freshly isolated” mouse cardiomyocytes cultured in 96-well-plates and exposed to the complexes at 100 μg/ml.

Methods

In vitro ischemic injury was induced in mouse neonatal primary cardiomyocyte culture by oxygen glucose deprivation (OGD) for 18 hours. Cardiomyocytes were incubated with different treatments of the rotomer complexes during 4 hours prior to the OGD induction. The test compounds were also present during the OGD insult and 24 h-recovery period. NAC was used as positive control of cardioprotection. After treatments, cells were simultaneously loaded with two fluorescent dyes showing optical compatibility, and were then analyzed with BD Pathway 855 (Becton Dickinson). By using the technology of high content screening (HCS), cell parameters associated with caspase 3/7 activation (indicative of pre-lethal cytotoxic effects and representative of different mechanism of toxicity) were measured at the single cells level, which allows high-throughput screening.

Primary cultures of cardiomyocytes were prepared from the hearts of Swiss mouse neonates P0-P2. Hearts were removed and enzymatically dissociated following instructions from Neonatal heart Dissociation Kit of Miltenyi Biotec. Then, cardiomyocytes were isolated following also the instructions of Miltenyi Biotec. Briefly, cells were isolated by depletion of non-target cells. Nontarget cells (fibroblasts and endothelial cells) are directly magnetically labeled with a cocktail of monoclonal antibodies conjugated with MACS microbeads. The magnetically labeled non-target cells were depleted by retaining them within a MCS column in the magnetic fields of a MACS separator, while the unlabeled cardiomyocytes passed through the column. Then, cardiomyocytes were counted and plated on fibronectin-coated wells with cardiac myocyte complete medium (1% CMGS component, 1% penicillin/streptomycin and 5% FBS).

### Caspase 3/7 Activation Assay:

Caspase 3/7 activation was determined using The CellEvent® Caspase-3/7 Green Detection Reagent which is intrinsically a non-fluorescent peptide that inhibits the ability of the dye to bind to DNA. However, after caspase-3/7 activation in apoptotic cells, the peptide is cleaved allowing the dye to bind to DNA producing a bright, fluorogenic response. Cell were stained with 5 μM reagent, washed 3 times and measured at 488 nm/530 nm Ex/Em. This dye permits the direct quantification of apoptotic cells. For all the rotomer complexes, a variation of at least 20% in fluorescence intensity or in the corresponding morphological parameter in relation to vehicle-treated cultures under OGD conditions was considered. In the present study, OGD toxicity is linked to an increase in caspase 3/7 activation. The preventive effects shown by rotomer complexes against OGD toxicity are associated with restoration of caspase 3/7 activity.

FIG. 8 shows the results of the Caspase 3/7 activation assay of positive and negative controls. Cardiomyocytes were incubated with NAC during 4 hours prior to the OGD induction and was also present during the OGD insult and 24 h-recovery period. After treatments. HCS assay was performed. Data points represent the mean+SD for each condition of number of apoptotic cells per nuclei.

FIG. 9 shows the results of the assay in which cardiomyocytes were incubated with the rotomer complexes during 4 hours prior to the OGD induction and were also present during the OGD insult and 24 h-recovery period. After treatments, HCS assay was performed. Data points represent the mean ±SD for each condition of number of apoptotic cells per nuclei. In FIG. 9, COM 1 is the hydroxymaleate anhydride complex of oltipraz: (−)-rotomer described in Example 2 above. COM 2 is the hydrochloride of oltipraz: (+) rotomer described in Example 1 above. COM 3 is the dimethyl phosphate complex of oltipraz: (−) rotomer described in Example 9 above, and COM 4 is the acetyl chloride complex of oltipraz: (+) rotomer described in Example 3 above. Data points represent the mean±SD for each condition of number of apoptotic cells per nuclei. As shown in FIG. 9, each of the rotomer complexes exhibited a significant degree of cardioprotection.

### Recitation of Aspects

1. A composition comprising a rotomeric excess of the (+) rotomer of 4-methyl-5-(pyrazin-2-yl)-3H-1,2-dithiole-3-thione (oltipraz, see FIG. 4A).

2. A composition according to aspect 1, wherein the composition comprises one or more oltipraz-complexes (e.g., one or more salts of oltipraz) haying a rotomeric excess of the (+) rotomer.

3. A composition according to aspect 2, wherein the one or more oltipraz-complexes comprise a salt resulting from addition of a group (e.g., reversible addition of HCl, alkyl halides or acyl halides) to the alpha nitrogen of oltipraz's pyrazinyl ring producing a N-substituted pyrazini urn ion and an associated anion.

4. A composition according to aspect 3, wherein the one or more oltipraz-complexes comprise at least one oltipraz-complex formed by the addition to the alpha nitrogen of oltipraz's pyrazinyl ring of one or more reactants selected from the group consisting of alkyl halide, alkenyl halide, alkynyl halide, acyl halide, benzoyl halide, and combinations thereof (e.g., one or more reactants selected from the group consisting of: HCl, methyl chloride, substituted or unsubstituted methyl, substituted or unsubstituted formyl, substituted or unsubstituted acetyl, substituted or unsubstituted benzoyl, and substituted or unsubstituted butyl). Such oltipraz-complexes may be formed by reaction of oltipraz with e.g., formyl chloride, acetyl chloride, benzoyl chloride, n-butyl iodide, methyl iodide, and combinations thereof. In such an aspect the formyl, acetyl, benzoyl and butyl groups may be associated with the nitrogen atoms at the 2 or 4 position of the pyrazine ring, and may bear one or more substituents selected from, for example, halogen (e.g., F or Cl) and/or lower alkyl (C1-C6 alkyl).

5. A composition according to any of aspects 1-4, wherein the rotomeric excess of the (+) rotomer is in a range selected from the group consisting of a 30-40% excess, a 40-50% excess, a 50-60% excess, a 60-70% excess, a 70-80% excess, and greater than 80% excess.

6. A composition comprising an oltipraz-complex wherein the oltipraz-complex has an excess of the (−) rotomer of 4-methyl-5-(pyrazin-2-yl)-3H-1,2-dithiole-3-thione (oltipraz).

7. A composition according to aspect 6, wherein the composition comprises one or more oltipraz-complexes having a rotomeric excess of the (−) rotomer.

8. A composition according to aspect 7, wherein at least one of the one or more oltipraz-complexes comprises a direct bond between the alpha nitrogen of the pyrazinyl ring and an electronegative atom.

9. A composition according to aspect 8, wherein the electronegative atom is selected from the group consisting of oxygen, sulfur, and phosphorus.

10. A composition according to any of aspects 6-9. wherein the one or more oltipraz-complexes comprise a hydroxymaleate oltipraz-complex.

11. A composition according to any of aspects 6-9, wherein the one or more oltipraz-complexes comprise one or more complexes formed by reacting oltipraz with one or more reactants selected from the group consisting of a carboxy acid or a salt thereof, a phosphoric acid or a salt thereof, an alkyl phosphate, an aryl phosphate, an alkyl sulfonic acid or a salt thereof, an aryl sulfonic acid or a salt thereof, a substituted or unsubstituted thiocarboxy acid or a salt thereof, and combinations of one or more of the foregoing, e.g., lactic acid, tartronic acid, isothionic acid, isoserine, 2-mercaptoethane sulfonic acid. taurine, propyl phosphonic acid, 2-aminoethylphosphonic acid, diethyl dithiophosphate, diethyl thiophosphate, di methyl phosphate.

12. A composition according to any of aspects 6-9, wherein the one or more oltipraz-complexes comprise one or more complexes formed by reacting oltipraz with one or more reactants selected from the group consisting of lactic acid, tartronic acid, isothionic acid, isoserine, 2-mercaptoethane sulfonic acid, taurine, propyl phosphonic acid, 2-aminoethylphosphonic acid, diethyl dithiophosphate, diethyl thiophosphate, di methyl phosphate, and combinations thereof

13. A composition according to any of aspects 6-12, wherein the rotomeric excess of the (−) rotomer is in a range selected from the group consisting of a 30-40% excess, a 40-50% excess, a 50-60% excess, a 60-65% excess, a 60-70% excess, a 70-80% excess, an 80-90% excess, and a 90-100% excess.

14. A pharmaceutical composition comprising at least one composition according to any of aspects 1-13. wherein the composition is suitable for administration to a human.

15. A pharmaceutical composition according to aspect 14, wherein the composition is in a. form suitable for topical administration.

16. A pharmaceutical composition according to aspect 15, wherein the composition is in a form suitable for rectal administration or in a form suitable for administration by inhalation.

17. A pharmaceutical composition according to aspect 14, wherein the composition is in a form suitable for subcutaneous, intramuscular, intrastemal, or intravenous injection.

18. A pharmaceutical composition according to aspect 14. wherein the composition is in a. form suitable for oral administration.

19. A pharmaceutical composition according to aspect 18, wherein the composition is in the form of a powder, pill, tablet, or contained within a capsule.

20. A pharmaceutical composition according to aspect 18, wherein the composition is in a liquid form.

21. A process for treating a human or non-human animal patient in need comprising administering to the patient a pharmaceutical composition according to any of aspects 14-20.

22. A process according to aspect 21, wherein the administration comprises an oral administration.

23. A process according to aspect 21, wherein the administration comprises a buccal administration.

24. A process for preventing, treating, ameliorating the symptoms of, lessening the severity of, and/or shortening the duration of mucositis for a human or non-human animal patient in need comprising administering to the patient a pharmaceutical composition according to any of aspects 18-20.

25. A process for preventing, treating, ameliorating the symptoms of, lessening the severity of, and/or shortening the duration of mucositis for a human or non-human animal patient in need comprising administering to the patient a pharmaceutical composition according to any of aspects 18-20.

26. A process for preventing, treating, ameliorating the symptoms of, lessening the severity of, and/or shortening the duration of mucositis for a human or non-human animal patient in need comprising orally administering to the patient a pharmaceutical composition according to any of aspects 18-20.

27. A process according to any of aspects 24-26, wherein the mucositis is oral mucositis.

28. A process according to any of aspects 24-26, wherein the mucositis is mucositis of the alimentary canal.

29. A process for preventing, treating, ameliorating the symptoms of, lessening the severity of, and/or shortening the duration of mucositis or dermatitis for a human or non-human animal patient in need comprising topically administering to the patient a composition according to aspect 15.

30. A process for preventing, treating, ameliorating the symptoms of, lessening the severity of, and/or shortening the duration of mucositis for a human or non-human animal patient in need comprising rectally administering a composition according to aspect 16.

31. A process for preventing, treating, ameliorating the symptoms of, lessening the severity of, and/or shortening the duration of mucositis for a human or non-human animal patient in need comprising administering by inhalation a composition according to aspect 16.

32. A process according to any of aspects 21-31, wherein the patient is undergoing radiation therapy.

33. A process for making ahydroxymaleate anhydride (−) rotomer of oltipraz, comprising the steps of:
(i) reacting oltipraz with (+)-diacetyl-L-tartaric anhydride in the presence of a dry polar solvent to form a reaction mixture; and
(ii) adding acetic acid to the reaction mixture.

34. A process for preventing, treating, ameliorating the symptoms of, lessening the severity and/or shortening the duration of a viral infection for a human or non-human animal patient in need comprising administering a pharmaceutical composition according to any of aspects 14-20.

35. A process for treating a human or non-human animal patient at risk of a viral infection to better withstand a viral infection comprising administering a pharmaceutical composition according to any of aspects 14-20.

36. A process according to aspect 34 or 35, wherein the patient has one or more of hypertension, decreased pulmonary function, or a respiratory disease or condition.

37. A process for treating a human or non-human animal patient at risk of a stroke injury and/or ischemia/reperfusion injury comprising administering a pharmaceutical composition according to an of aspects 14-20.

38. A process for preventing, treating, ameliorating the symptoms of, lessening the severity and/or shortening the duration of a stroke injury and/or ischemia/reperfusion injury for a human or non-human animal patient in need comprising administering a pharmaceutical composition according to any of aspects 14-20.

39. A process for preventing, treating, ameliorating the symptoms of, lessening the severity and/or shortening the duration of, or to prevent, lessen or reduce the effects and/or symptoms associated with cardiac ischemia for a human or non-human animal patient in need comprising administering a pharmaceutical composition according to any of aspects 14-20.

40. A process for treating a human or non-human animal patient at risk of cardiac ischemia injury comprising administering a pharmaceutical composition according to any of aspects 14-20.

41. A process according to any of aspects 34-40, wherein the administration comprises an oral administration.

42. A process according to any of aspects 34-40, wherein the administration comprises a buccal administration.

43. A process according to any of aspects 34-40, wherein the administration comprises an intravenous administration.

44. A process according to any of aspects 34-40, wherein the administration comprises administration by inhalation,

45. A process according to any of aspects 34-44, wherein the pharmaceutical composition comprises a hydroxy maleate oltipraz-complex.

46. A process according to any of aspects 21-32, wherein the pharmaceutical composition comprises a hydroxymaleate oltipraz-complex.

47. A pharmaceutical composition according to any of aspects 14-20, wherein the pharmaceutical composition comprises a hydroxymaleate oltipraz-complex.

48. A composition comprising one or more oltipraz-complexes wherein the oltipraz-complexes have an excess of the (+) rotomer of 4-methyl-5-(pyrazin-2-yl)-3H-1,2-dithiole-3-thione (oltipraz) for use in a method of
(i) preventing, treating, ameliorating the symptoms of, lessening the severity and/or shortening the duration of mucositis for a human or non-human animal patient in need thereof:
(ii) treating a patient undergoing radiation therapy;
(iii) preventing, treating, ameliorating the symptoms of, lessening the severity and/or shortening the duration of a viral infection for a human or non-human animal patient in need thereof, wherein optionally the patient has one or more of hypertension, decreased pulmonary function, or a respiratory disease or condition;
(iv) treating a human or non-human animal patient at risk of a viral infection to better withstand a viral infection, wherein optionally the patient has one or more of hypertension, decreased pulmonary function, or a respiratory disease or condition;
(v) treating a human or non-human animal patient at risk of a stroke injury and/or ischemia/reperfusion injury;
(vi) preventing, treating, ameliorating the symptoms of, lessening the severity and/or shortening the duration of a stroke injury and/or ischemia/reperfusion injury for a human or non-human animal patient in need thereof;
(vii) preventing, treating, ameliorating the symptoms of, lessening the severity and/or shortening the duration of, or to prevent, lessen or reduce the effects and/or symptoms associated with cardiac ischemia for a human or non-human animal patient in need thereof;
(viii) treating a human or non-human animal patient at risk of cardiac ischemia injury; or
(ix) preventing, treating, ameliorating the symptoms of, lessening the severity and/or shortening the duration of mucositis or dermatitis for a human or non-human animal patient in need thereof;
   the method comprising administering a composition comprising a rotomeric excess of the (+) rotomer of 4-methyl-5-(pyrazin-2-yl)-31-1-1,2-dithiole-3-thione to the patient.

49. A composition according to aspect 48, wherein the composition comprises one or more oltipraz-complexes having a rotomeric excess of the (+) rotomer.

50. A composition according to aspect 49, wherein the one or more oltipraz-complexes comprise a salt resulting from addition of a group to the alpha nitrogen of oltipraz's pyrazinyl ring producing a N-substituted pyrazinium ion and an associated anion.

51. A composition according to aspect 50, wherein the one or more oltipraz-complexes comprise at least one oltipraz-complex formed from the reaction of oltipraz and one or more reactants selected from the group consisting of alkyl halide, alkenyl halide, alkynyl halide, acyl halide, benzoyl halide, and combinations thereof.

52. A composition according to any of aspects 48-51, wherein the rotomeric excess of the (+) rotomer is in a range selected from the group consisting of a 30-40% excess, a 40-50% excess, a 50-60% excess, a 60-70% excess, a 70-80% excess, and greater than 80% excess.

53. A composition comprising one or more oltipraz-complexes wherein the oltipraz-complexs have an excess of the (−) rotomer of 4-methyl-5-(pyrazin-2-yl)-3H-1,2-dithiole-3-thione (oltipraz) for use in a method of
(i) preventing, treating, ameliorating the symptoms of, lessening the severity and/or shortening the duration of mucositis for a human or non-human animal patient in need thereof
(ii) treating a patient undergoing radiation therapy;
(iii) preventing, treating, ameliorating the symptoms of, lessening the severity and/or shortening the duration of a viral infection for a human or non-human animal patient in need thereof, wherein optionally the patient has one or more of hypertension, decreased pulmonary function, or a respiratory disease or condition;
(iv) treating a human or non-human animal patient at risk of a viral infection to better withstand a viral infection, wherein optionally the patient has one or more of hypertension, decreased pulmonary function, or a respiratory disease or condition;
(v) treating a human or non-human animal patient at risk of a stroke injury and/or ischemia/reperfusion injury;
(vi) preventing, treating, ameliorating the symptoms of, lessening the severity and/or shortening the duration of a stroke injury and/or ischemia/reperfusion injury for a human or non-human animal patient in need thereof;
(vii) preventing, treating, ameliorating the symptoms of, lessening the severity and/or shortening the duration of, or to prevent, lessen or reduce the effects and/or symptoms associated with cardiac ischemia for a human or non-human animal patient in need thereof;
(viii) treating a human or non-human animal patient at risk of cardiac ischemia injury; or
(ix) preventing, treating, ameliorating the symptoms of, lessening the severity and/or shortening the duration of mucositis or dermatitis for a human or non-human animal patient in need thereof;
   the method comprising administering a composition comprising a rotomeric excess of the (−) rotomer of 4-methyl-5-(pyrazin-2-yl)-3H-1,2-dithiole-3-thione to the patient.

54. A composition according to aspect 53, wherein the composition comprises one or more oltipraz-complexes having a rotomeric excess of the (−) rotomer.

55. A composition according to aspect 54, wherein at least one of the one or more oltipraz-complexes comprises a direct bond between the alpha nitrogen of the pyrazinyl ring and an electronegative atom.

56. A composition according to aspect 55. wherein the electronegative atom is selected from the group consisting of oxygen, sulfur, and phosphorus.

57. A composition according to any of aspects 53-56, wherein the one or more oltipraz-complexes comprise a hydroxymaleate oltipraz-complex.

58. A composition according to any of aspects 53-56, wherein the one or more oltipraz-complexes comprise one or more complexes formed by reacting oltipraz with one or more reactants selected from the group consisting of a carboxy acid or a salt thereof, a phosphoric acid or a salt thereof, an alkyl phosphate, an aryl phosphate, an alkyl sulfonic acid or a salt thereof, an aryl sulfonic acid or a salt thereof, a substituted or unsubstituted thiocarboxy acid or a salt thereof, and combinations of one or more of the foregoing.

59. A composition according to any of aspects 53-56, wherein the one or more oltipraz-complexes comprise one or more complexes formed by reacting oltipraz with one or more reactants selected from the group consisting of lactic acid, tartronic acid, isothionic acid, isoserine, 2-mercaptoethane sulfonic acid, taurine, propyl phosphoric add, 2-aminoethylphosphonic acid, diethyl dithiophosphate, diethyl thiophosphate, dimethyl phosphate, and combinations thereof.

60. A composition according to any of aspects 53-59, wherein the rotomeric excess of the (−) rotomer is in a range selected from the group consisting of a 30-40% excess, a 40-50% excess, a 50-60% excess, a 60-65% excess, a 60-70% excess, a 70-80% excess, an 80-90% excess, and a 90-100% excess.

61. The composition according to any of aspects 48-60, wherein the method is a method of preventing, treating, ameliorating the symptoms of, lessening the severity and/or shortening the duration of mucositis for a human or non-human animal patient in need thereof.

62. The composition according to any of aspects 48-60, wherein the method is a method of treating a patient undergoing radiation therapy.

63. The composition according to any of aspects 48-60, wherein the method is a method of preventing, treating, ameliorating the symptoms of, lessening the severity and/or shortening the duration of a viral infection for a human or non-human animal patient in need thereof, wherein optionally the patient has one or more of hypertension, decreased pulmonary function, or a respiratory disease or condition.

64. The composition according to any of aspects 48-60, wherein the method is a method of treating a human or non-human animal patient at risk of a viral infection to better withstand a viral infection, wherein optionally the patient has one or more of hypertension, decreased pulmonary function, or a respiratory disease or condition.

65. The composition according to any of aspects 48-60, wherein the method is a method of treating a human or non-human animal patient at risk of a stroke injury and/or ischemia/reperfusion injury.

66. The composition according to any of aspects 48-60, wherein the method is a method of preventing, treating, ameliorating the symptoms of, lessening the severity and/or shortening the duration of a stroke injury and/or ischemia/reperfusion injury for a human or non-human animal patient in need thereof.

67. The composition according to any of aspects 48-60, wherein the method is a method of preventing, treating, ameliorating the symptoms of, lessening the severity and/or shortening the duration of, or to prevent, lessen or reduce the effects and/or symptoms associated with cardiac ischemia for a human or non-human animal patient in need thereof.

68. The composition according to any of aspects 48-60, wherein the method is a method of treating a human or non-human animal patient at risk of cardiac ischemia injury.

69. The composition according to any of aspects 48-60, wherein the method is a method of preventing, treating, ameliorating the symptoms of, lessening the severity and/or shortening the duration of mucositis or dermatitis for a human or non-human animal patient in need thereof.

70. The composition according to any of aspects 48-60, wherein the method is a method of treating oral mucositis.

71. The composition according to any of aspects 48-60, wherein the method is a method of treating mucositis of the alimentary canal.

72. The composition according to any of aspects 48-71, wherein the composition is suitable for administration to a human.

73. The composition according to any of aspects 48-72, wherein the composition is in a form suitable for topical administration and administering is conducted topically.

74. The composition according to any of aspects 48-72, wherein the composition is in a form. suitable for rectal administration and administering is conducted rectally, or in a form suitable for administration by inhalation and the administering is conducted by inhalation.

75. The composition according to any of aspects 48-72, wherein the composition is in a form suitable for subcutaneous, intramuscular, intrasternal, or intravenous injection and the administering is conducted by injection.

76. The composition according to any of aspects 48-72, wherein the composition is in a form suitable for oral administration and the administering is conducted by the oral administration.

77. The composition according to aspect 76, wherein the administering is conducted by a buccal administration.

78. The composition according to aspect 76, wherein the composition is in the form of a powder, pill, tablet, or contained within a capsule.

79. The composition according to aspect 76, wherein the composition is in a liquid form.

80. The composition according to any of aspects 48-79, wherein the composition is a pharmaceutical composition comprising one or more pharmaceutically acceptable excipients.

81. The composition according to any of aspects 53-79, wherein the composition is a pharmaceutical composition comprising one or more pharmaceutically acceptable excipients and a hydroxymaleate oltipraz-complex.

1. A method of treatment comprising administering to a patient in need thereof a composition comprising one or more oltipraz-complexes wherein the oltipraz-complexes have an excess of the (+) rotomer of 4-methyl-5-(pyrazin-2-yl)-3H-1,2-dithiole-3-thione (oltipraz) for
(i) preventing, treating, ameliorating the symptoms of, lessening the severity and/or shortening the duration of mucositis for a human or non-human animal patient in need thereof
(ii) treating a patient undergoing radiation therapy;
(iii) preventing, treating, ameliorating the symptoms of, lessening the severity and/or shortening the duration of a viral infection for a human or non-human animal patient in need thereof, wherein optionally the patient has one or more of hypertension, decreased pulmonary function, or a respiratory disease or condition;
(iv) treating a human or non-human animal patient at risk of a viral infection to better withstand a viral infection, wherein optionally the patient has one or more of hypertension, decreased pulmonary function, or a respiratory disease or condition;
(v) treating a human or non-human animal patient at risk of a stroke injury and/or ischemia/reperfusion injury;
(vi) preventing, treating, ameliorating the symptoms of, lessening the severity and/or shortening the duration of a stroke injury and/or ischemia/reperfusion injury for a human or non-human animal patient in need thereof;
(vii) preventing, treating, ameliorating the symptoms of, lessening the severity and/or shortening the duration of, or to prevent, lessen or reduce the effects and/or symptoms associated with cardiac ischemia for a human or non-human animal patient in need thereof;
(viii) treating a human or non-human animal patient at risk of cardiac ischemia injury; or
(ix) preventing, treating, ameliorating the symptoms of, lessening the severity and/or shortening the duration of mucositis or dermatitis for a human or non-human animal patient in need thereof.

2. The method according to claim 1, wherein the composition comprises one or more oltipraz-complexes having a rotomeric excess of the (+) rotomer.

3. The method according to claim 2, wherein the one or more oltipraz-complexes comprise a salt resulting from addition of a group to the alpha nitrogen of oltipraz's pyrazinyl ring producing a N-substituted pyrazinium ion and an associated anion.

4. The method according to claim 3, wherein the one or more oltipraz-complexes comprise at least one oltipraz-complex formed from the reaction of oltipraz and one or more reactants selected from the group consisting of alkyl halide, alkenyl halide, alkynyl halide, acyl halide, benzoyl halide, and combinations thereof.

5. The method according to claim 1, wherein the rotomeric excess of the (+) rotomer is in a range selected from the group consisting of a 30-40% excess, a 40-50% excess, a 50-60% excess, a 60-70% excess, a 70-80% excess, and greater than 80% excess.

6. A method of treatment comprising administering to a patient in need thereof a composition comprising one or more oltipraz-complexes wherein the oltipraz-complexes have an excess of the (−) rotomer of 4-methyl-5-(pyrazin-2-yl)-3H-1,2-dithiole-3-thione (oltipraz) for
(i) preventing, treating, ameliorating the symptoms of, lessening the severity and/or shortening the duration of mucositis for a human or non-human animal patient in need thereof
(ii) treating a patient undergoing radiation therapy;
(iii) preventing, treating, ameliorating the symptoms of, lessening the severity and/or shortening the duration of a viral infection for a human or non-human animal patient in need thereof, wherein optionally the patient has one or more of hypertension, decreased pulmonary function, or a respiratory disease or condition;
(iv) treating a human or non-human animal patient at risk of a viral infection to better withstand a viral infection, wherein optionally the patient has one or more of hypertension, decreased pulmonary function, or a respiratory disease or condition;
(v) treating a human or non-human animal patient at risk of a stroke injury and/or ischemia/reperfusion injury;
(vi) preventing, treating, ameliorating the symptoms of, lessening the severity and/or shortening the duration of a stroke injury and/or ischemia/reperfusion injury for a human or non-human animal patient in need thereof;
(vii) preventing, treating, ameliorating the symptoms of, lessening the severity and/or shortening the duration of, or to prevent, lessen or reduce the effects and/or symptoms associated with cardiac ischemia for a human or non-human animal patient in need thereof;
(viii) treating a human or non-human animal patient at risk of cardiac ischemia injury; or (ix) preventing, treating, ameliorating the symptoms of, lessening the severity and/or shortening the duration of mucositis or dermatitis for a human or non-human animal patient in need thereof.

7. The method according to claim 6, wherein the composition comprises one or more oltipraz-complexes having a rotomeric excess of the (−) rotomer.

8. The method according to claim 7, wherein at least one of the one or more oltipraz-complexes comprises a direct bond between the alpha nitrogen of the pyrazinyl ring and an electronegative atom.

9. The method according to claim 8, wherein the electronegative atom is selected from the group consisting of oxygen, sulfur, and phosphorus.

10. The method according to claim 6, wherein the one or more oltipraz-complexes comprise a hydroxymaleate oltipraz-complex.

11. The method according to claim 6, wherein the one or more oltipraz-complexes comprise one or more complexes formed by reacting oltipraz with one or more reactants selected from the group consisting of a carboxy acid or a salt thereof, a phosphoric acid or a salt thereof, an alkyl phosphate, an aryl phosphate, an alkyl sulfonic acid or a salt thereof, an aryl sulfonic acid or a salt thereof, a substituted or unsubstituted thiocarboxy acid or a salt thereof, and combinations of one or more of the foregoing.

12. The method according to claim 6, wherein the one or more oltipraz-complexes comprise one or more complexes formed by reacting oltipraz with one or more reactants selected from the group consisting of lactic acid, tartronic acid, isothionic acid, isoserine, 2-mercaptoethane sulfonic acid, taurine, propyl phosphonic acid, 2-aminoethylphosphonic acid, diethyl dithiophosphate, diethyl thiophosphate, dimethyl phosphate, and combinations thereof.

13. The method according to claim 6, wherein the rotomeric excess of the (−) rotomer is in a range selected from the group consisting of a 30-40% excess, a 40-50% excess, a 50-60% excess, a 60-65% excess, a 60-70% excess, a 70-80% excess, an 80-90% excess, and a 90-100% excess.

14. The method according to 13 claim 6, wherein the composition is suitable for administration to a human.

15. The method according to claim 6, wherein the is in a form suitable for topical administration and the administering is conducted topically, or the composition is in a form suitable for rectal administration and the administering is conducted rectally, or the composition is in a form suitable for administration by inhalation and the administering is conducted by inhalation.

16. The method according to claim 6, wherein the composition is in a form suitable for subcutaneous, intramuscular, intrasternal, or intravenous injection and the administering is conducted by injection.

17. The method according to claim 6, wherein the composition is in a form suitable for oral administration and the administering is conducted by the oral administration.

18. The method according to claim 17, wherein the composition is in the form of a liquid, powder, pill, tablet, or contained within a capsule.

19. The method according to claim 6, wherein the method is a method of treating oral mucositis or mucositis of the alimentary canal.

20. The method according to claim 6, wherein the composition is a pharmaceutical composition comprising one or more pharmaceutically acceptable excipients and a hydroxymaleate oltipraz-complex.
